(19) United States
(12) Patent Application Publication
(10) Pub. No.: US 2023/0381482 A1
(43) Pub. Date: Nov. 30, 2023

(54) Introducer Tool for an Aspiration Catheter

(71) Applicant: Neuravi Limited, Galway (IE)

(72) Inventors: Patrick GRIFFIN, Galway (IE); David VALE, Galway (IE); Karl KEATING, Galway (IE); Thomas O'MALLEY, Galway (IE); Lucy CARRAGHER, Ontario (CA); Ronald KELLY, Galway (IE); Aidan DUFFY, Galway (IE); Declan LEE, Galway (IE); Paulo RATOLA, Galway (IE)

(21) Appl. No.: 18/312,184

(22) Filed: May 4, 2023

Related U.S. Application Data
(60) Provisional application No. 63/347,526, filed on May 31, 2022.

(51) Int. Cl. A61M 39/06; A61M 25/06

(52) U.S. Cl. CPC A61M 39/0693; A61M 25/0662; A61B 2017/22079

(57) ABSTRACT

An introducer tool for use with a vascular entryway system and method. While a radially self-expanding distal section of an aspiration catheter is in a radially non-compressed state, an aspiration catheter is pre-assembled into a flared proximal section of the introducer tool. Together as a single unit, the introducer tool with the aspiration catheter pre-assembled therein is introduced into a hemostasis valve and a tapered guide sheath luer. The aspiration catheter is pushed through the introducer tool. While traversing the compressing section of the lumen of the shaft of the introducer tool and/or the tapered inner profile of a tapered guide sheath luer, the radially self-expanding distal section of the aspiration catheter is radially compressed to be receivable in a lumen of a guide sheath catheter. While the radially self-expanding distal section is radially compressed, the aspiration catheter is slid into the lumen of the guide sheath catheter.

### CROSS REFERENCE TO RELATED PATENT APPLICATIONS

This patent application claims the benefit of U.S. Provisional Application Ser. No. 63/347,526, filed May 31, 2022, which is herein incorporated by reference it its entirety.

### FIELD OF THE DISCLOSURE

The present disclosure relates to an introducer tool for an aspiration catheter, for example, a funnel catheter. In particular, the disclosure is directed to an introducer tool for guiding an aspiration catheter through a hemostasis valve and into the lumen of a guide sheath catheter.

### DESCRIPTION OF RELATED ART

Aspiration catheters are conventionally used during intravascular treatments or procedures such as a thrombectomy procedure to apply a vacuum pressure to capture a target occlusion in a vessel. Aspiration catheters may be used in combination with a mechanical thrombectomy device (e.g., stent retriever). When passing an aspiration catheter through a hemostasis valve and into a guide catheter, it would be beneficial to protect the relatively soft, open mouthed, flexible distal tip susceptible to damage if snagged on an edge while being guided into the lumen of the guide sheath catheter. It also being advantageous to minimize friction, maintain the wetted outer diameter of the aspiration catheter to prevent the hydrophilic coating from drying out, minimizing surface contact, and preventing axial loading of the aspiration catheter.

It is desirable to develop an improved introducer tool for guiding an aspiration catheter through the hemostasis valve and into the lumen of the guide sheath catheter that addresses all these concerns.

### SUMMARY OF THE DISCLOSURE

An aspect of the present disclosure is directed to an introducer tool for guiding an aspiration catheter (e.g., funnel catheter) through a hemostasis valve and into the lumen of a guide sheath catheter.

Another aspect of the present disclosure is directed to an introducer tool for guiding an aspiration catheter (e.g., funnel catheter) through a hemostasis valve and into the lumen of a guide sheath catheter while protecting a relatively soft, open mouthed, flexible distal tip susceptible to damage if snagged on an edge while being guided into the lumen of the guide sheath catheter.

Yet another aspect of the present disclosure relates to an introducer tool for guiding an aspiration catheter (e.g., funnel catheter) through a hemostasis valve and into the lumen of a guide sheath catheter while maintaining the wetted outer diameter of the funnel catheter to prevent the hydrophilic coating from drying out.

While still other aspects of the present disclosure relate to an introducer tool for guiding an aspiration catheter (e.g., funnel catheter) through a hemostasis valve and into the lumen of a guide sheath catheter while minimizing friction, maintaining the wetted outer diameter of the aspiration catheter to prevent the hydrophilic coating from drying out, minimizing surface contact, and preventing axial loading of the aspiration catheter.

### BRIEF DESCRIPTION OF THE DRAWING

The foregoing and other features of the present disclosure will be more readily apparent from the following detailed description and illustrative drawings wherein like reference numbers refer to similar elements throughout the several views and in which:

FIG. 1A is an axial/longitudinal cross-sectional view of an vascular entryway system including a first example of an introducer tool in accordance with the present disclosure having a shaft member with a straight (i.e., cylindrical—uniform in both inner and outer diameters) distal section and a lumen defined therethrough in which is inserted unhindered a funnel catheter whose distal section is in a flared (i.e., open biased, radially enlarged, non-compressed radially of maximum outer diameter) state; the introducer tool and funnel catheter pre-assembled therein together as a single unit is introduced into an assembly (including a hemostasis valve attached to a guide sheath catheter via a tapered guide sheath lure/hub);

FIG. 1B is an axial/longitudinal cross-sectional view of the vascular entryway system of FIG. 1A depicting the distal section of the funnel catheter in a radially compressed state of reduced outer diameter after directly physically engaging with the inner walls/profile of the tapered guide sheath luer/hub able to be accommodated in the smaller inner diameter lumen of the guide sheath catheter;

FIG. 1C is an axial/longitudinal cross-sectional view of a vascular entryway system with a modified configuration of the introducer tool of FIG. 1A having a shaft member with a straight (i.e., cylindrical—uniform in both inner and outer diameters) distal section and a lumen defined therethrough having an inner diameter sized to radially compress (i.e., reduce in outer diameter) a flared distal section of the funnel catheter when inserted therein; the introducer tool and funnel catheter pre-assembled therein together as a single unit is introduced into an assembly (including a hemostasis valve attached to a guide sheath catheter via a tapered guide sheath lure/hub);

FIG. 1D is an axial/longitudinal cross-sectional view of the vascular entryway system of FIG. 1C depicting the distal section of the funnel catheter in a radially compressed state of reduced outer diameter after passing through the distal section of the introducer tool able to be accommodated in the smaller inner diameter lumen of the guide sheath catheter without directly physically contacting the inner wall/profile of the tapered guide sheath lure/hub;

FIG. 2A is an axial/longitudinal cross-sectional view of a vascular entryway system with a second example of an introducer tool in accordance with the present disclosure having a shaft member with a tapered only in outer diameter distal section and a lumen defined therethrough in which is inserted unhindered a funnel catheter whose distal section is in a flared (i.e., open biased, radially enlarged, non-compressed radially of maximum outer diameter) state; the introducer tool and funnel catheter pre-assembled therein together as a single unit is introduced into an assembly (including a hemostasis valve attached to a guide sheath catheter via a tapered guide sheath lure/hub);

FIG. 2B is an axial/longitudinal cross-sectional view of the vascular entryway system of FIG. 2A depicting the distal section of the funnel catheter in a radially compressed state of reduced outer diameter able to be accommodated in the smaller inner diameter lumen of the guide sheath catheter;

FIG. 3A is an axial/longitudinal cross-sectional view of a third example of an introducer tool in accordance with the present disclosure having a shaft member with a lumen defined therethrough; a distal section of the shaft member is tapered both in its inner diameter and outer diameters;

FIG. 3B is an axial/longitudinal cross-sectional view of a funnel catheter whose distal section is in a flared (i.e., open biased, radially enlarged, non-compressed radially of maximum outer diameter) state inserted unhindered into the lumen of the shaft member of the introducer tool of FIG. 3A prior to entering the tapered both in its inner and outer diameters distal section;

FIG. 3C is an axial/longitudinal cross-sectional view of the vascular entryway system with the introducer tool of FIG. 3A wherein the flared (i.e., open biased, radially enlarged, non-compressed radially of maximum outer diameter) distal section of the funnel catheter is depicted unhindered (non-compressed radially) in the introducer tool;

FIG. 3D is an axial/longitudinal cross-sectional view of the vascular entryway system of FIG. 3C depicting the distal section of the funnel catheter radially compressed (reduced in outer diameter) after traversing through the tapered distal section of the introducer tool able to be accommodated in the inner diameter of the lumen of the guide sheath catheter without directly physically contacting the tapered inner wall of the guide sheath luer/hub;

FIG. 4A is an axial/longitudinal cross-sectional view of a modified example of the introducer tool of FIG. 3A having a distal section tapered both in inner and outer diameters and a plurality of flushing ports defined in the outer wall of the shaft member;

FIG. 4B is an axial/longitudinal cross-sectional view of a funnel catheter whose distal section is in a flared (i.e., open biased, radially enlarged, non-compressed radially of maximum outer diameter) state traversing unhindered the lumen of the introducer tool of FIG. 4A prior to entering its distal section tapered both in inner and outer diameters;

FIG. 4C is an axial/longitudinal cross-sectional view of the vascular entryway system with an introducer tool of FIG. 4A pre-assembled with the funnel catheter therein together as a single unit introduced into an assembly (including a hemostasis valve attached to a guide sheath catheter via a tapered guide sheath lure/hub);

FIG. 4D is an axial/longitudinal cross-sectional view of the vascular entryway system of FIG. 4C depicting the distal section of the funnel catheter compressed radially (i.e., reduced in outer diameter) after traversing the tapered both in inner and outer diameter distal section of the introducer tool and accommodated within the lumen of the guide sheath catheter without directly physically contacting the tapered inner wall/profile of the guide sheath luer/hub;

FIG. 5A is an axial/longitudinal cross-sectional view of a modified example of the introducer tool of FIG. 3A whose distal section is tapered both in inner and outer diameters with an axial/longitudinal slit defined in the outer wall of the shaft member splitting the introducer tool for easy removal from around the funnel catheter;

FIG. 5B is an axial/longitudinal cross-sectional view of a further modification of the introducer tool of FIG. 5A with a series of flushing ports aligned with the axial/longitudinal slit;

FIG. 5C is an axial/longitudinal cross-sectional view of another modified example of the introducer tool of FIG. 3A with a single longitudinal slot defined in the outer wall of the shaft member the axial/longitudinal edges of which are radially separated from one another to form an opening allowing the passage of fluid therethrough;

FIG. 5D is an axial/longitudinal cross-sectional view of still another modified example of the introducer tool of FIG. 3A with a single axial/longitudinal slit defined in the outer wall of the shaft member the longitudinal edges of which radially overlap each other;

FIG. 6A is an axial/longitudinal cross-sectional view of a yet another modified example of the introducer tool of FIG. 3A whose distal section is tapered both in inner and outer diameters with one or more axial/longitudinal extending weakened regions (e.g., perforations) in the outer wall of the shaft member allowing one or more axial/longitudinal sections between adjacent weakened regions to be independently peeled away while the funnel catheter remains in the assembly;

FIG. 6B is an axial/longitudinal cross-sectional view of a further modification of the introducer tool of FIG. 6A with a series of flushing ports aligned with the weakened region (e.g., perforations) defined in the outer wall of the shaft member;

FIG. 7A is an axial/longitudinal cross-sectional view of a vascular entryway system with an alternative of the introducer tool of FIG. 3A to include a funnel/cone shape proximal section in which is nestable in an identical funnel/cone shape proximal section of a funnel catheter when fully inserted into the assembly;

FIG. 7B is an axial/longitudinal cross-sectional view of the vascular entryway system of FIG. 7A depicting further advancement (i.e., partial insertion) in a distal direction of the funnel catheter with its distal section radially compressed (reduced in outer diameter) after passing through the tapered inner diameter of the distal section of the introducer tool and accommodated in the lumen of the guide sheath catheter;

FIG. 7C is an axial/longitudinal cross-sectional view of FIG. 7B depicting the funnel catheter advanced still further in a distal direction with its distal section fully inserted in the lumen of the guide sheath catheter; over insertion of the funnel catheter in the assembly being prevented by the proximal section funnel/cone of the funnel catheter physically seated in the proximal section funnel/cone of the introducer tool;

FIG. 8A is a side view of still another example of the introducer tool including a unidirectional transition section prohibiting over insertion in the guide sheath tapered luer/hub thereby preventing compression of the distal tip and narrowing of the lumen; the unidirectional transition section providing a tapered or stepped interface between: (i) a non-insertable straight (i.e., cylindrical—uniform in both inner and outer diameter) section the inner diameter of which is able to accommodate unhindered therein the distal end of the funnel catheter in a flared (i.e., open biased, radially enlarged, non-compressed radially of maximum outer diameter) state; and (ii) an insertable straight (i.e., cylindrical—uniform in both inner and outer diameter) section having an outer diameter smaller relative to the non-insertable straight (cylindrical—uniform in both inner and outer diameter) section is receivable in the lumen of the guide sheath catheter; wherein the flared distal section of the funnel catheter is compressed radially as it travels through the insertable straight section of the introducer tool;

FIG. 8B is an axial/longitudinal cross-sectional view of a vascular entryway system including an introducer tool including a unidirectional transition section prohibiting over insertion in the guide sheath tapered luer/hub thereby preventing compression of the distal end/tip; the unidirectional transition section providing a tapered or stepped interface between: (i) a non-insertable straight (i.e., cylindrical—uniform in both inner and outer diameter) section the inner diameter of which is able to accommodate unhindered therein the flared (i.e., open biased, radially enlarged, non-compressed radially of maximum outer diameter) distal section of the funnel catheter; and (ii) an insertable straight (i.e., cylindrical—uniform in both inner and outer diameter) section the outer diameter of which is smaller relative to the non-insertable straight (i.e., cylindrical—uniform in both inner and outer diameter) section; depicting the distal section of the funnel catheter in a flared, open biased state advanceable unhindered (i.e., non-compressed radially) as it travels through both the non-insertable and the insertable straight sections;

FIG. 8C is an axial/longitudinal cross-sectional view of a vascular entryway system with an introducer tool including a bidirectional transition section prohibiting over insertion in the guide sheath tapered luer/hub thereby preventing compression of the distal tip; the bidirectional transition section providing a tapered or step-down interface in opposite directions between: (i) a non-insertable straight (i.e., cylindrical—uniform in both inner and outer diameter) section; and (ii) an insertable straight (i.e., cylindrical—uniform in both inner and outer diameter) section having an outer diameter equal to that of the non-insertable straight section; depicting the distal section of the funnel catheter in a flared (i.e., open biased, radially enlarged, non-compressed radially of maximum outer diameter) state advanceable unhindered as it travels therethrough both the non-insertable and the insertable straight sections;

FIG. 8D is an axial/longitudinal cross-sectional view of a vascular entryway system with another introducer tool including a unidirectional transition section providing a tapered or stepped interface between: (i) a proximal straight (i.e., cylindrical—uniform in both inner and outer diameter) section the inner diameter of which is able to accommodate unhindered therein the flared (i.e., open biased, radially enlarged, non-compressed radially of maximum outer diameter) distal section of the funnel catheter; and (ii) a distal straight (i.e., cylindrical—uniform in both inner and outer diameter) section the outer diameter of which is smaller relative to the proximal straight (i.e., cylindrical—uniform in both inner and outer diameter) section; depicting the distal section of the funnel catheter in a flared, open biased state advanceable unhindered (i.e., non-compressed radially) as it travels through the proximal straight section (prior to being compressed radially (reduced in outer diameter) when traversing the distal straight (cylindrical) section); the proximal section of the funnel catheter including a stiffer proximal region X;

FIG. 8E is an axial/longitudinal cross-sectional view of a vascular entryway system with an introducer tool including a unidirectional transition section providing a tapered or stepped interface between: (i) a proximal straight (i.e., cylindrical—uniform in both inner and outer diameter) section the inner diameter of which is able to accommodate unhindered therein the flared (i.e., open biased, radially enlarged, non-compressed radially of maximum outer diameter) distal section of the funnel catheter; and (ii) a distal straight (i.e., cylindrical—uniform in both inner and outer diameter) section the outer diameter of which is smaller relative to the proximal straight (i.e., cylindrical—uniform in both inner and outer diameter) section; depicting the distal section of the funnel catheter in a flared, open biased state advanceable unhindered (i.e., non-compressed radially) as it travels through the proximal straight section (prior to being compressed radially (reduced in outer diameter) when traversing the distal straight (cylindrical) section); the longer length of the proximal straight (cylindrical) section of the introducer tool in comparison to that of FIG. 8D providing additional support to the more flexible region of the funnel catheter disposed distally of the stiffer proximal region X;

FIG. 8F is an axial/longitudinal cross-sectional view of a vascular entryway system with still another introducer tool the inner and outer profiles of which has a continuous taper to its proximal flared section;

FIG. 9 is an axial/longitudinal cross-sectional view of a vascular entryway system with the introducer tool of FIG. 3A together with the funnel catheter pre-assembled therein together as a single unit advanced through an assembly (comprising a hemostasis valve connected to a guide sheath catheter by a tapered guide sheath lure/hub) and a removable extension holder secured about the flared/cone proximal section of the introducer tool providing additional length to protect the distal soft, flexible section of the funnel catheter when advancing the tip through the introducer tool and removable to allow full insertion of the funnel catheter length into the assembly;

FIG. 10 is an axial/longitudinal cross-sectional view of the vascular entryway system of FIG. 3C illustrating undesirable compression of the distal end/tip and narrowing of the lumen of the introducer tool resulting from direct physical contact with the tapered inner wall of the guide sheath luer/hub resulting from over insertion in the assembly;

FIG. 11A is a side view of an exemplary introducer tool having a radially expandable compression force absorption component comprising radially expandable pleats preventing compression of the distal end/tip and narrowing of the lumen when subject to excessive force (i.e., over insertion), wherein the radially expandable pleats are depicted in a non-deployed (i.e., axially/longitudinally non-compressed state not subject to over insertion) state;

FIG. 11B is an axial/longitudinal cross-sectional view of a vascular entryway system including the introducer tool of FIG. 11A along with the funnel catheter pre-assembled therein together as a unit inserted into the assembly (comprising the hemostasis valve connected to the guide sheath catheter via the tapered guide sheath luer/hub); wherein the radially expandable pleats are depicted in a non-deployed (i.e., axially/longitudinally non-compressed state not subject to over insertion) state;

FIG. 11C is a side view of the introducer tool of FIG. 11A, wherein the radially expandable pleats are depicted in a deployed (i.e., axially/longitudinally compressed state subject to over insertion) state;

FIG. 11D is an axial/longitudinal cross-sectional view of a vascular entryway system including the introducer tool of FIG. 11C along with the funnel catheter pre-assembled therein together as a unit inserted into the assembly (comprising the hemostasis valve connected to the guide sheath catheter via the tapered guide sheath luer/hub); wherein the radially expandable pleats are depicted in a deployed (i.e., axially/longitudinally compressed state subject to over insertion) state;

FIG. 12A is a side view of an exemplary introducer tool having a radially expandable compression force absorption component preventing compression of the distal end/tip when subject to excessive force (over insertion), wherein the radially expandable compression force absorption component comprises radially expandable arms formed in areas between adjacent axial/longitudinal slits defined in the shaft of the introducer tool; wherein the radially expandable arms are depicted in a non-deployed (i.e., axially/longitudinally non-compressed state not subject to over insertion having a uniform minimum outer diameter) state;

FIG. 12B is an axial/longitudinal cross-sectional view of a vascular entryway system with the introducer tool of FIG. 12A with the funnel catheter pre-assembled therein together as a unit inserted into the assembly (comprising the hemostasis valve connected to the guide sheath catheter via the tapered guide sheath luer/hub; wherein the radially expandable arms are depicted in a non-deployed (i.e., axially/longitudinally non-compressed state not subject to over insertion) state;

FIG. 12C is a side view of the introducer tool of FIG. 12A, wherein the radially expandable arms are depicted in a deployed (i.e., axially/longitudinally compressed state subject to over insertion having a non-uniform maximum outer diameter) state;

FIG. 12D axial/longitudinal cross-sectional view of the vascular entryway system with the introducer tool of FIG. 12C along with the funnel catheter pre-assembled therein together as a unit inserted into the assembly (comprising the hemostasis valve connected to the guide sheath catheter via the tapered guide sheath luer/hub; wherein the radially expandable arms are depicted in a deployed (i.e., axially/longitudinally compressed state subject to over insertion having a non-uniform maximum outer diameter) state;

FIG. 13A is a side view of an exemplary introducer tool having a radially expandable compression force absorption component preventing compression of the distal end/tip when subject to excessive force (over insertion), wherein the radially expandable compression force absorption component comprises angled radially expandable arms formed in areas between adjacent angled slits defined in the shaft of the introducer tool; wherein the angled radially expandable arms are depicted in a non-deployed (i.e., axially/longitudinally non-compressed state not subject to over insertion having a uniform minimum outer diameter) state;

FIG. 13B is an axial/longitudinal cross-sectional view of a vascular entryway system with the introducer tool of FIG. 13A with the funnel catheter pre-assembled therein together as a unit inserted into the assembly (comprising the hemostasis valve connected to the guide sheath catheter via the tapered guide sheath luer/hub; wherein the angled radially expandable arms are depicted in a non-deployed (i.e., axially/longitudinally non-compressed state not subject to over insertion) state;

FIG. 13C is a side view of the introducer tool of FIG. 13A, wherein the angled radially expandable arms are depicted in a deployed (i.e., axially/longitudinally compressed state subject to over insertion having a non-uniform maximum outer diameter) state;

FIG. 13D axial/longitudinal cross-sectional view of the vascular entryway system with the introducer tool of FIG. 13C along with the funnel catheter pre-assembled therein together as a unit inserted into the assembly (comprising the hemostasis valve connected to the guide sheath catheter via the tapered guide sheath luer/hub; wherein the angled radially expandable arms are depicted in a deployed (i.e., axially/longitudinally compressed state subject to over insertion having a non-uniform maximum outer diameter) state;

FIG. 14A is a side view of an exemplary introducer tool having a radially expandable compression force absorption component preventing compression of the distal end/tip when subject to excessive force (over insertion), wherein the radially expandable compression force absorption component comprises radially expandable arms formed in areas between adjacent series of axial/longitudinal slits in an alternating pattern defined in the shaft of the introducer tool; wherein the radially expandable arms are depicted in a non-deployed (i.e., axially/longitudinally non-compressed state not subject to over insertion having a uniform minimum outer diameter) state;

FIG. 14B is an axial/longitudinal cross-sectional view of a vascular entryway system with the introducer tool of FIG. 14A with the funnel catheter pre-assembled therein together as a unit inserted into the assembly (comprising the hemostasis valve connected to the guide sheath catheter via the tapered guide sheath luer/hub; wherein the radially expandable arms are depicted in a non-deployed (i.e., axially/longitudinally non-compressed state not subject to over insertion) state;

FIG. 14C is a side view of the introducer tool of FIG. 14A, wherein the radially expandable arms are depicted in a deployed (i.e., axially/longitudinally compressed state subject to over insertion having a non-uniform maximum outer diameter) state;

FIG. 14D axial/longitudinal cross-sectional view of the vascular entryway system with the introducer tool of FIG. 14C along with the funnel catheter pre-assembled therein together as a unit inserted into the assembly (comprising the hemostasis valve connected to the guide sheath catheter via the tapered guide sheath luer/hub; wherein the radially expandable arms are depicted in a deployed (i.e., axially/longitudinally compressed state subject to over insertion having a non-uniform maximum outer diameter) state;

FIG. 15A is a side view of an exemplary introducer tool having a radially expandable compression force absorption component comprising a material with a lower stiffness susceptible to radially outward expansion (i.e., bulging) when subject to compression forces relative to the material comprising the remaining portions of the shaft member of the introducer tool thereby preventing compression of the distal end/tip and narrowing of the lumen when subject to excessive force (over insertion), wherein the radially expandable compression force absorption component is depicted in a non-deployed (i.e., axially/longitudinally non-compressed state not subject to over insertion) state;

FIG. 15B is an axial/longitudinal cross-sectional view of a vascular entryway system including the introducer tool of FIG. 15A along with the funnel catheter pre-assembled therein together as a unit inserted into the assembly (comprising the hemostasis valve connected to the guide sheath catheter via the tapered guide sheath luer/hub); wherein the radially expandable compression force absorption component is depicted in a non-deployed (i.e., axially/longitudinally non-compressed state not subject to over insertion) state;

FIG. 15C is a side view of the introducer tool of FIG. 15A, wherein the material having a lower stiffness susceptible to radially outward expansion (i.e., bulging) when subject to compression forces relative to the material comprising the remaining portions of the shaft member of the introducer tool is depicted in a deployed (i.e., axially/longitudinally compressed state subject to over insertion) state;

FIG. 15D is an axial/longitudinal cross-sectional view of a vascular entryway system including the introducer tool of FIG. 15C along with the funnel catheter pre-assembled therein together as a unit inserted into the assembly (comprising the hemostasis valve connected to the guide sheath catheter via the tapered guide sheath luer/hub); wherein the radially expandable compression force absorption component is depicted in a deployed (i.e., axially/longitudinally compressed state subject to over insertion) state;

FIG. 16A is perspective view of a distal end of an introducer tool in accordance with the present disclosure illustrating axial/longitudinal internal ribs projecting radially inward along the inner wall of the lumen;

FIG. 16B is the perspective view of one half of the introducer tool of FIG. 16A in an axial/longitudinal direction to illustrate the axial/longitudinal internal ribs projecting radially inward along the inner wall of a distal section of the lumen;

FIGS. 17A-17G are radial cross-sectional views through the distal section of the introducer tool depicting various non-circular geometric shapes of the lumen; where FIG. 17E is a radial cross-sectional view through the internal ribs along lines 17(E)-17(E) in FIG. 16A;

FIG. 18A is an axial/longitudinal cross-sectional view of a vascular entryway system including an introducer tool having an axially/longitudinally contractable section comprising telescopic tubes depicted in an axially/longitudinally expanded (non-contracted) state (i.e., maximum axial/longitudinal length L1);

FIG. 18B is an axial/longitudinal-sectional view of the vascular entryway system of FIG. 18A with the telescopic tubes of the axially/longitudinally contractable section of the introducer tool depicted in an axially/longitudinally contracted state (i.e., minimum axial/longitudinal length L2);

FIG. 19A is an axial/longitudinal cross-sectional view of a vascular entryway system including an introducer tool having an axially/longitudinally contractable section comprising a plurality of bellows depicted in an axially/longitudinally expanded (non-contracted) state (i.e., maximum axial/longitudinal length L1);

FIG. 19B is an axial/longitudinal cross-sectional view of the vascular entryway system of FIG. 19A with the plurality of bellows of the axially/longitudinally contractable section of the introducer tool depicted in an axially/longitudinally contracted state (i.e., minimum axial/longitudinal length L2);

FIG. 20A is a side view of still another example of the introducer tool in accordance with the present disclosure having two transition sections and the proximal section peelably separable apart along a longitudinal/axial direction to form handles/tabs for manipulating the device;

FIG. 20B is a proximal end view of the introducer tool of FIG. 20A;

FIG. 20C is an axial/longitudinal cross-sectional view of the introducer tool of FIG. 20A alone lines 20(A)-20(A);

FIG. 20D is an enlarged axial/longitudinal cross-sectional view of section 20(D)-20(D) of FIG. 20A, illustrating the distal tapered end having a uniform inner diameter and a tapered outer diameter;

FIG. 20E is a perspective view of the introducer tool of FIG. 20A;

FIG. 21A is a side view of yet another example of a semi-split introducer tool in accordance with the present disclosure with one of the peelably separable apart handles/tabs cut away to show the inner region of the remaining handle/tab;

FIG. 21B is an enlarged side view of a portion of the unsplit section “A” of the introd4ucer tool of FIG. 21A identified as section 21(B);

FIG. 21C is an enlarged side view of transition section of the introducer tool of FIG. 21A i5dentified as section 21(C);

FIG. 22A is a side view of still another example of a semi-split introducer tool in accordance with the present disclosure with one of the peelably separable apart handles/tabs cut away to show the interior region of the remaining handle/tab; wherein the introducer tool has a proximal section of enhanced rigidity;

FIG. 22B is an enlarged cross-sectional view through the introducer tool of FIG. 22A identified as section 22(B);

FIG. 22C is an enlarged cross-sectional view through the introducer tool of FIG. 22A identified as section 22(C);

FIG. 23A is yet another example of a semi-split introducer tool in accordance with another aspect of the present disclosure wherein the rigid proximal section is maximized by eliminating the peelably separable apart handles/tabs;

FIG. 23B is an enlarged axial/longitudinal cross-sectional view through the introducer tool of FIG. 23A identified as section 23(B);

FIG. 23C is an enlarged axial/longitudinal cross-sectional view through the introducer tool of FIG. 23A identified as section 23(C); and

FIG. 23D depicts the introducer tool of FIG. 23A with the aspirator catheter and strain relief accommodated in the lumen of the introducer tool and over insertion prevented by the proximal hub.

### DETAILED DESCRIPTION OF THE DISCLOSURE

In the description, the terms “distal” or “proximal” are used in the following description with respect to a position or direction relative to the treating physician or medical interventionalist. “Distal” or “distally” are a position distant from or in a direction away from the physician or interventionalist. “Proximal” or “proximally” or “proximate” are a position near or in a direction toward the physician or medical interventionist. The terms “occlusion”, “clot” or “blockage” are used interchangeably.

Aspiration catheters (e.g., a funnel catheter) have a radially self-expanding, open mouthed, open biased, flared distal section (including the distal end) with an integral shaft extending in a proximal direction therefrom. In order to track through the tortuous vasculature, the flared distal section of the aspiration catheter is more conformable and the distal shaft section is more flexible relative to that of the proximal shaft. Preferably, the axial length of the flexible distal shaft section of the funnel catheter is 1 cm-30 cm and the conformable flared distal section is 2 to 10 mm. Such increased stiffness (less flexibility) of the proximal shaft of the funnel catheter may be achieved by an embedded braid pattern or a polymer jacket having a desired durometer. When passing an aspiration catheter through a hemostasis valve and into a guide catheter an introducer tool may be employed to protect the relatively soft, open mouthed, radially expandable, conformable flared distal section susceptible to snag on an edge while being advanced and flexible distal shaft section susceptible to buckle under axial compressive loads as the flared distal section is collapsed and advanced through a guide catheter. Design of the introducer tool also addresses the challenge posed in inserting the radially self-expanding, flared (i.e., open biased, open mouthed, radially enlarged, non-compressed radially of maximum outer diameter) distal flared section of the aspiration catheter into the smaller inner diameter lumen of the guide sheath catheter. Further, in the field, the physician preferably pre-assembles the aspiration catheter into the introducer tool outside the body which together as a single unit may then be quickly inserted through the hemostasis valve and into the guide sheath catheter in order to minimize blood loss. Keeping the outer diameter of the aspiration catheter wetted to activate the hydrophilic coating is challenging because the open design of the introducer tool allows any flushing fluid to quickly drain away causing the hydrophilic coating to dry out. In the case of a catheter having a uniform outer profile/diameter this is not an issue as the outer diameter of the catheter is less than the inner diameter of the introducer tool and the guide sheath allowing flushing therebetween. However, for an aspiration catheter, the outer diameter of the self-expanding open mouth (enlarged/flared distal end) is larger than the conventional inner diameter of the guide sheath. If a conventional introducer tool is used with a uniform inner diameter close to that of the lumen of the guide sheath catheter, the enlarged/flared distal section of the aspiration catheter has to be compressed radially (i.e., collapsed/reduced in outer diameter) in order to be passable therethrough potentially trapping air in the flared catheter if pre-loaded with an auxiliary device (e.g., microcatheter/mechanical thrombectomy device). However, with the difficulties described associated with flushing the introducer tool and in keeping the hydrophilic coating wetted, friction may buildup restricting passage therethrough of the enlarged/flared distal section of the aspiration catheter as it passes through the introducer tool. Furthermore, advancement of the aspiration catheter through the introducer tool may cause the enlarged/flared distal section to collapse. Moreover, a compressive resistance is produced when advancing the flexible enlarged/flared distal section of the aspiration catheter through the introducer tool so another concern when overcoming this force is the risk of potentially damaging the shaft of the introducer tool if gripped too tightly. The present inventive introducer tool suitable for use with an aspiration catheter addresses these concerns. By way of example, the present inventive introducer tool is illustrated and described for use with a funnel catheter; however, the present inventive introducer tool is suitable for use with other types of aspiration catheters.

For every configuration of the present inventive introducer tool illustrated and described herein, prior to being introduced in the body (i.e., prior to being inserted in the assembly (i.e., comprising the hemostasis valve connected to the guide sheath catheter via a tapered guide sheath luer/hub), the funnel catheter is pre-assembled in the introducer tool while outside of the body. Thereafter, the introducer tool with the funnel catheter pre-assembled therein together as a single unit is inserted into the assembly comprising a hemostasis valve attached to a guide sheath catheter via a tapered guide sheath lure/hub. An inner wall/profile of the tapered guide sheath lure/hub is tapered at its distal end having a minimum inner diameter preferably substantially equal or close to that of the inner diameter of the lumen of the guide sheath catheter.

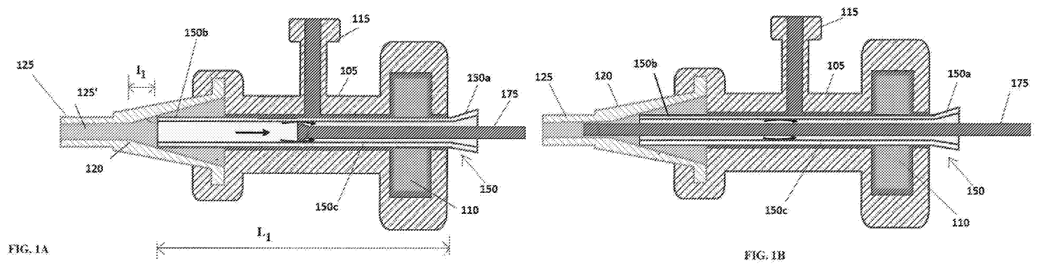

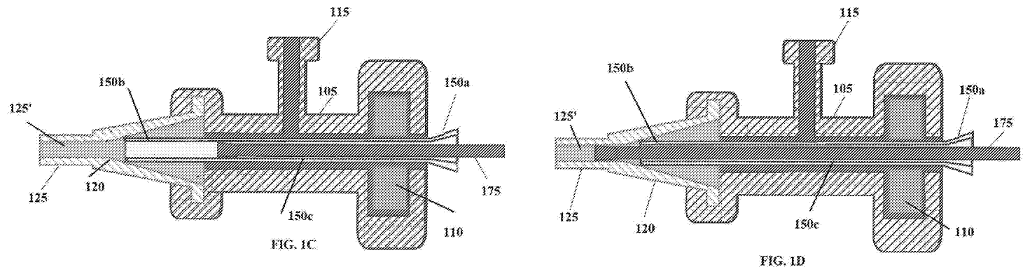

Referring to the first example shown in FIG. 1A, the assembly comprises the hemostasis valve 105 having a hemostasis seal 110 and a hemostasis side port 115. Attached to the distal end of the hemostasis valve 105 is a tapered guide sheath luer/hub 120 the inner wall/profile of which is tapered having a minimum inner diameter substantially equal to the inner diameter of the lumen 125′ of the guide sheath catheter 125 connected thereto.

In the example illustrated in FIG. 1A, the present inventive introducer tool 150 has a cone/flared proximal section 150a integral with a shaft disposed distally thereof. The shaft of the introducer tool includes a straight (i.e., cylindrical, uniform in both inner and outer diameters) section 150c terminating in a straight (i.e., cylindrical, uniform both in inner and outer diameters) distal section 150b as a single integral unit or connected to one another. Along its entire length (in an axial/longitudinal direction) the inner diameter of the lumen of the shaft of the introducer tool 150 is larger than the outer diameter of the distal section of the funnel catheter 175 while in a flared (i.e., open biased, radially enlarged, non-compressed radially of maximum outer diameter) state. Accordingly, the funnel catheter 175 while its distal section is in a flared (i.e., open biased, radially enlarged, non-compressed radially of maximum outer diameter) state is advanceable unhindered (i.e., free from radial compression) within the introducer tool 150. The introducer tool 150 with the funnel catheter 175 pre-assembled therein together as a single unit is advanced in a distal direction through the assembly (i.e., comprising the hemostasis valve 105, tapered guide sheath catheter lure/hub 120, and guide sheath catheter 125) until the distal end/tip of the introducer tool 150 is in direct physical contact with (i.e., abuts) the tapered inner wall/profile of the guide sheath lure/hub 120. As denoted in FIG. 1A by the directional arrows pointing in the proximal direction, while being advanced in a distal direction through the introducer tool 150, the hydrophilic coating of the funnel catheter 175 is automatically hydrated by back pressure of fluid (e.g., blood) passing in a proximal direction around the distal section while in a flared (i.e., open biased, radially enlarged, non-compressed radially of maximum outer diameter) state. Any clearance or space greater than zero between the outer diameter of the distal section of the funnel catheter 175 while in a flared (i.e., open biased, radially enlarged, non-compressed radially of maximum outer diameter) state and the inner diameter of the introducer tool 150 may be provided to permit back pressure fluid to flow in a proximal direction around the flared distal section of the funnel catheter 175. Preferably, a nominal clearance range of approximately 0.002″-0.005″ is provided to allow for manufacturing tolerances between the inner diameter of the introducer tool 150 and the outer diameter of the distal section of the funnel catheter 175 while in a flared (i.e., open biased, radially enlarged, non-compressed radially of maximum outer diameter) state. After exiting from the distal end/tip of the introducer tool 150 when directly physically engaging with the tapered inner wall/profile of the guide sheath lure/hub 120, the flared distal section of the funnel catheter 175 radially collapses/compresses (i.e., reduces in outer diameter) sufficient to pass through the lumen 125′ of the guide sheath catheter 125 (having a smaller lumen than the inner diameter of the straight distal section 150b of the introducer tool 150).

Regardless of the particular example of the introducer tool, when selecting a desired axial/longitudinal length (from a proximal end/tip to an opposite distal end/tip) one or more factors may be taken into consideration: (i) shorter in length is easier for the interventionalist to manipulate; (ii) sufficient length is desirable to grip between the thumb and forefinger when the introducer tool with the funnel catheter pre-assembled therein together as a single unit is inserted through the hemostasis valve; and (iii) longer in length provides more reinforcement to the relatively soft/flexible/expandable distal section of the funnel catheter when pushing to collapse (i.e., reduce in outer diameter) the flared distal section prior to entering the smaller inner diameter of the lumen of the guide sheath catheter. Preferably, the axial/longitudinal length of the introducer tool from the proximal end/tip to the opposite end/tip is in a range of 4 cm-50 cm. In addition, every configuration of the present inventive introducer tool illustrated and described herein preferably has a cone/flared proximal section 150a (including the proximal end/tip) with both the outer and inner diameters being largest at its proximal end/tip to allow for easy, unhindered insertion of the funnel catheter without radially compressing/collapsing/reducing the flared (i.e., open biased, radially enlarged, non-compressed radially of maximum outer diameter) distal section. The cone/flared proximal section 150a also acts as a stop to prevent over insertion of the introducer tool 150 into the hemostasis valve 105.

The lumen of the shaft of the introducer tool 150 shown in FIGS. 1A & 1B has an inner diameter larger than the inner diameter of the guide sheath catheter 125. Accordingly, the funnel catheter 175 while its distal section is in a flared (i.e., open biased, radially enlarged, non-compressed radially of maximum outer diameter) state is advanceable unhindered (i.e., free from radial compression) through the lumen of the introducer tool 150 from its proximal end/tip to its opposite distal end/tip. Upon emerging from the distal end/tip of the introducer tool 150 the flared distal section of the funnel catheter 175 is radially compressed (i.e., reduced in outer diameter) upon directly physically engaging with the tapered inner walls/profile of the guide sheath hub/luer 120 until sufficiently compressed radially (i.e., reduced in outer diameter) to be receivable within the lumen 125′ of the guide sheath catheter 125. However, it is also possible for the inner diameter of the shaft 150c of the introducer tool 150 having a straight section 150c and the straight distal section 150b (similar to that of FIGS. 1A & 1B) to be closer in size (preferably, substantially equal in size) to that of the inner diameter of the lumen 125′ of the guide sheath catheter 125. In such case the outer diameter of the flared (i.e., open biased, non-compressed radially of maximum outer diameter) distal section of the funnel catheter 175 is greater than the inner diameter of the lumen through the straight (i.e., cylindrical, uniform in both inner and outer diameters) sections 150b, 150c of the introducer tool. Accordingly, when advanced through the straight sections 150b, 150c of the introducer tool, the flared distal section of the funnel catheter 175 is radially compressed (i.e., reduced in outer diameter) (FIG. 1C). As a result of the inner diameter at the distal section 150b of the introducer tool 150 being equal to the inner diameter of the lumen 125′ of the guide sheath catheter 125, the funnel catheter 175 is directly transferred from the introducer tool 150′ into the guide sheath catheter 125 without the flared distal section being further compressed radially or directly physical engaging with the tapered inner walls of the guide sheath hub/luer 120 (FIG. 1D).

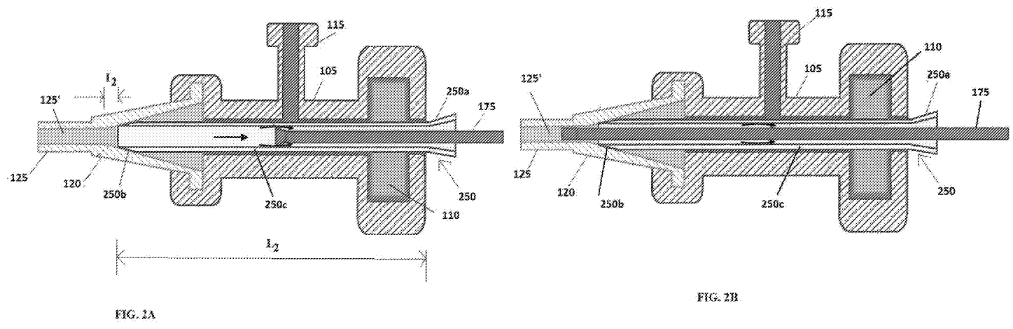

Insertion into the assembly (i.e., the hemostasis valve 105, tapered guide sheath luer/hub 120, and guide sheath catheter 125) of the introducer tool 150 having straight (i.e., cylindrical, non-tapered, uniform in both outer and inner diameter) sections 150b, 150c (FIGS. 1A & 1B) ceases when its distal end/tip is in direct physical contact with the tapered internal walls of the guide sheath lure/hub 120. A modified structure of the present inventive introducer tool is shown in FIGS. 2A & 2B allowing for further insertion. In a comparison of the configurations in FIGS. 1A & 1B with that of FIGS. 2A & 2B, all features are the same with one notable exception. The distal section 150b (including the distal tip/end) of the introducer tool 150 in FIGS. 1A and 1B has a straight (i.e., cylindrical, non-tapered, uniform in both outer and inner diameter) configuration, whereas in the alternative example of FIGS. 2A & 2B, only the outer diameter of the distal section 250b of the introducer tool 250 is tapered. The inner diameter of the distal section 250b of the introducer tool 250 remains uniform (i.e., non-tapered) thereby allowing unhindered (i.e., free from radial compression) travel of the funnel catheter with its distal section in a flared (i.e., open biased, non-compressed radially of maximum outer diameter) state through the entire axial/longitudinal length of the introducer tool 250. Tapering of the outer profile of the distal section 250b of introducer tool 250 is preferably identical to that of the tapered inner wall of the guide sheath lure/hub 120 in order to minimize the gap between the distal face of the introducer tool and the inner tapered surface of the guide catheter lure/hub. Like the introducer tool 150 of FIGS. 1A & 1B, once again introducer tool 250 of FIGS. 2A& 2B has a sufficiently large inner diameter to accommodate unhindered (i.e., free from radial compression) insertion therein of the funnel catheter 175 with the distal section thereof in a flared (i.e., open biased, non-compressed radially of maximum outer diameter) state. Its tapered outer profile/outer diameter of the distal section 250b allows the introducer tool 250 to be inserted/pushed further in a distal direction into the assembly (compared with the configuration of FIGS. 1A & 1B) before coming into direct physical engagement with the tapered inner wall of the guide sheath lure/hub 120 providing smoother (i.e., minimizing obstructions) passage/transfer of the funnel catheter 175 through the interface between the introducer tool 250 and the guide sheath catheter 125.

The straight (i.e., cylindrical, non-tapered, uniform outer profile) distal section 150b of the introducer tool 150 in FIGS. 1A & 1B produces a stepped interface, transition or edge between the distal end of the introducer tool 150 and the tapered inner profile of the guide sheath lure/hub 120. When a force is applied in a proximal direction (i.e., pushing) on the proximal end of the funnel catheter 175, as the funnel emerges out from the distal end of the introducer tool 150 and encounters the tapered inner wall of the guide sheath lure/hub 120 the stepped interface, transition or edge therebetween provides a space (I₁) allowing undesirable slight additional radial expansion (i.e., flaring more than when disposed in the introducer tool 150) of the distal section of the funnel catheter 175. This undesirable stepped interface, transition or edge is minimized or eliminated altogether in the tapered outer profile distal section 250b of the introducer tool 250 in FIGS. 2A & 2B in which the transfer of the funnel catheter 175 between the introducer tool 250 and guide sheath luer/hub 120 occurs without flaring (i.e., no additional expansion radially outward of the distal section relative to that while unhindered in the introducer tool 250). A smooth transfer (i.e., without additional radially outward expansion of the distal section) of the funnel catheter 175 from the introducer tool 250 to the guide sheath luer/hub 120 is therefore provided by the tapered outer profile of the distal section 250b of the introducer tool 250 in FIGS. 2A & 2B. The tapered outer profile design of the distal section 250b of the introducer tool 250 in FIGS. 2A & 2B has the additional advantage of maximizing in an axial/longitudinal direction the extent of insertion (I1>I2) or in other words the axial/longitudinal length (L1<L2) of insertion of the introducer tool 250 into the assembly (i.e., hemostasis valve 105, tapered guide sheath luer/hub 120, and guide sheath catheter 125). Maximizing the axial/longitudinal length of insertion of the introducer tool 250 into the assembly (i.e., hemostasis valve 105, tapered guide sheath luer/hub 120, and guide sheath catheter 125) advantageously maximizes distance travel in an axial/longitudinal direction of the funnel catheter 175 unhindered while its distal section is maintained in a flared (i.e., open biased, radially enlarged, non-compressed radially of maximum outer diameter) state. In other words, by maximizing unhindered distance travel (axially/longitudinally in the distal direction) through the introducer tool 250, subsequent distance travel of the funnel catheter 175 upon emerging out from the distal end/tip of the introducer tool and transitioning to a constrained/compressed (i.e., non-flared, non-open biased of reduced outer diameter) state is advantageously minimized.

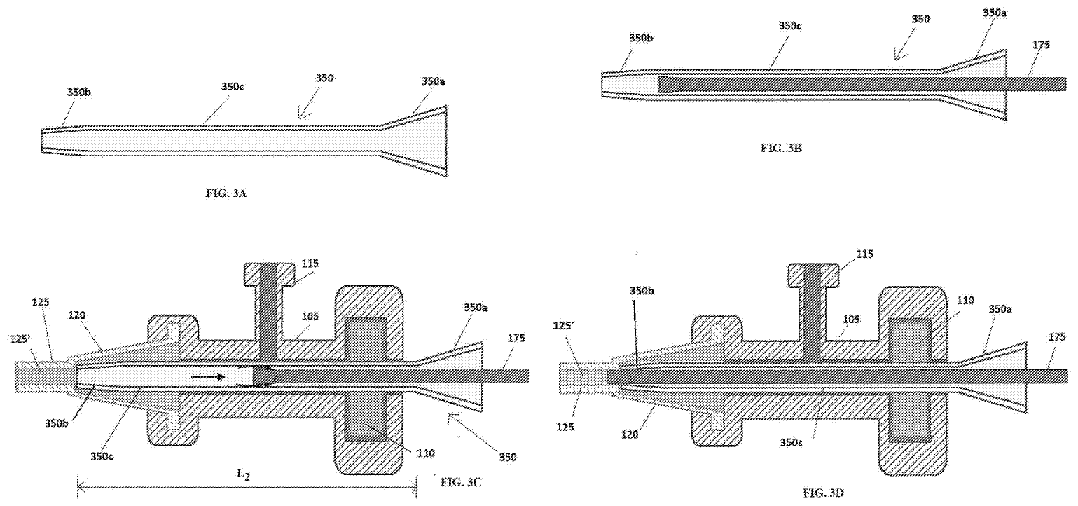

While FIGS. 3A-3D show still yet another example of the introducer tool 350 whose distal section 350b is tapered, non-uniform in both its outer and inner diameters with the smallest respective diameters (both inner diameter and outer diameter) arranged at the distal end/tip. As with the other examples, the introducer tool 350 (FIG. 3A) has a flared proximal section 350a for easy and unhindered (i.e., free from radial compression) pre-assembly therein of the funnel catheter 175 while its distal section is in a flared (i.e., open biased, radially enlarged, non-compressed radially of maximum outer diameter) state. Similar to that in FIGS. 2A & 2B, the distal section 350b of the introducer tool 350 in FIG. 3A also has a tapered outer diameter (i.e., smallest at the distal end/tip) maximizing insertion length (L2) in an axial/longitudinal direction into the assembly (i.e., hemostasis valve 105, tapered guide sheath luer/hub 120, and guide sheath catheter 125). While traveling through the tapered (non-uniform) inner diameter of the distal section 350b of the introducer tool 350, the distal section of the funnel catheter 175 is radially compressed/collapsed/reduced in outer diameter. At its distal end/tip the inner diameter of the introducer tool 350 is preferably substantially equal to that of the inner diameter of the lumen 125′ of the guide sheath catheter 125 providing smooth, unobstructed travel or passage of the funnel catheter 175 between the two components 350, 125. Between the respective proximal and distal sections 350a,350b of the introducer tool 350 is a straight (i.e., cylindrical, non-tapered, uniform in both outer and inner diameters) section 350c, the inner diameter of which is larger than a maximum outer diameter of the distal section of the funnel catheter 175 in a flared (i.e., open biased, radially enlarged, non-compressed radially of maximum outer diameter) state allowing unhindered (i.e., free from radial compression) travel of the funnel catheter 175 therethrough. FIG. 3B illustrates the pre-assembled unhindered (i.e., non-radially compressed) insertion of the funnel catheter 175 while its distal section is in a flared (i.e., open biased, radially enlarged, non-compressed radially of maximum outer diameter) state traversing the proximal section 350a and straight (i.e., cylindrical, uniform in both inner and outer diameters) section 350c (prior to entering the tapered distal section 350b) of the introducer tool 350. Pre-assembly of the funnel catheter 175 in the introducer tool 350 outside the body in this manner prior to insertion of the introducer tool 350 into the hemostasis valve 105 minimizes the risk of blood loss through the hemostasis valve 105. The straight (i.e., cylindrical uniform in both inner and outer diameters) section 350c of the introducer tool 350 with an inner diameter larger than that of the outer diameter of the funnel catheter 175 while its distal section is in a flared (i.e., open biased, radially enlarged, non-compressed radially of maximum outer diameter) state allows unhindered travel therethrough mitigating friction even when the hydrophilic coating of the funnel catheter is dried out. When the introducer tool 350 with the funnel catheter 175 pre-assembled therein together as a single unit is inserted into the hemostasis valve 105 (FIG. 3C), the back pressure of blood (as denoted by the directional arrows around the flared distal section of the funnel catheter) auto flushes/hydrates the lumen of the introducer tool 350 ensuring full hydration (i.e., wetting) of the hydrophilic coating minimizing friction of the funnel catheter 175 while optimizing advancement into the guide sheath catheter 125. As the funnel catheter 175 passes through the tapered inner diameter of the distal section 350b of the introducer tool 350 its flared distal section is radially compressed/collapsed/reduced in outer diameter to that substantially equal in size to the inner diameter of the lumen 125′ of the guide sheath catheter 125 allowing smooth passage or transfer therebetween (FIG. 3D). Preferably, the axial/longitudinal length of the introducer tool 350 is minimized to maximize the length of insertion of the funnel catheter 175 through the hemostasis valve 105 and into the guide sheath catheter 125.

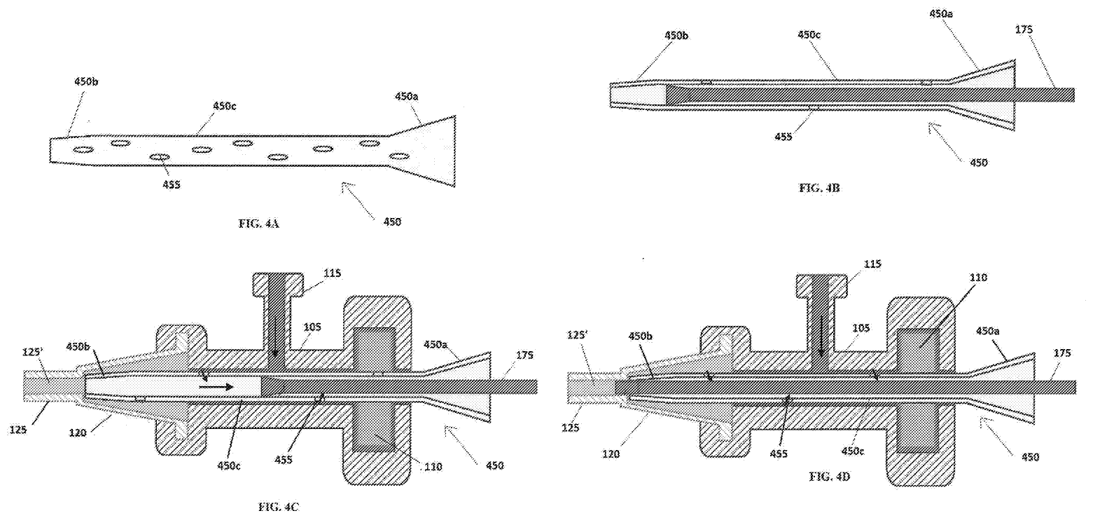

While still another modification of the introducer tool 450 is represented in FIGS. 4A-4D. This design of the introducer tool 450 has the same tapered distal section 450b (i.e., tapered both inner and outer diameters) as that in FIGS. 3A-3D but differs therefrom by having a plurality of venting or flushing ports (i.e., pores, holes, openings) 455 defined in its outer surface in fluid communication with the axial/longitudinal lumen. Any number of one or more flushing ports 455 are defined in the introducer tool 450 that allow entrained air to exit/vent therethrough as the funnel catheter 175 is advanced in a distal direction. Fluid (e.g., back pressure of blood and/or positive saline flush introduced through the side port 115 of the hemostasis valve 105) also passes through the flushing ports 455 of the introducer tool 450. Flushing ports 455 may be arranged, as desired (e.g., randomly, helical, aligned in an axial/longitudinal direction, aligned radially, offset radially). Location of the flushing ports may be anywhere from the proximal end/tip to the distal end/tip of the introducer tool (i.e., including the proximal section 450a, the straight (cylindrical) section 450c, and/or the distal section 450b). While selecting the number, arrangement and size of each of the flushing ports 455 maintaining the structural strength and integrity of the introducer tool is taken into consideration. The diameter of each flushing port (i.e., pore) and number are selected to allow blood cells to pass therethrough without shear stress damaging the cell. Preferably, the diameter of each flushing port is ≥ approximately 50 um. Operation of the introducer tool 450 is the same as that described above with respect to FIG. 3A-3D. Specifically, FIG. 4B illustrates a pre-assembly or insertion (i.e., unhindered, non-compressed radially) of the funnel catheter 175 in the proximal section 450a and straight (cylindrical) section 450c of introducer tool 450 while its distal section is in a flared (i.e., open biased, radially enlarged, non-compressed radially of maximum outer diameter) state. Pre-assembly of the funnel catheter 175 in the introducer tool 450 outside the body prior to insertion as a single unit together into the hemostasis valve 105 in this manner minimizes the risk of blood loss through the hemostasis valve 105. The straight (i.e., cylindrical, uniform in both inner and outer diameter) section 450c of the introducer tool 450 with an inner diameter larger than that of the outer diameter of the distal section of the funnel catheter 175 while in a flared (i.e., open biased, radially enlarged, non-compressed radially of maximum outer diameter) state allows unhindered (i.e., free from radial compression) travel therethrough. Such unhindered passage, mitigates against high friction between the components even under the circumstance of the hydrophilic coating of the funnel catheter drying out after flushing/wetting and prior to insertion in the introducer tool. When the pre-assembled introducer tool 450 and the funnel catheter 175 is advanced through the assembly (FIG. 4C), back pressure of blood and/or positive flushing with saline via the side port 115 of the hemostasis valve auto flushes the lumen of the introducer tool 450 via the flushing ports 455. As a result of this flushing, full hydration of the hydrophilic coating is ensured while minimizing friction of the funnel catheter 175 and optimizing advancement through the guide sheath catheter 125. As the funnel catheter 175 travels through the tapered inner diameter of the distal section 450b of the introducer tool 450 its flared distal section is radially compressed/collapsed/reduced in outer diameter to that substantially equal in size to the inner diameter of the lumen of the guide sheath catheter 125 allowing smooth passage or transfer therebetween (FIG. 4D). A drawback associated with the use of flushing ports 455 is the risk that when the funnel catheter 175 with its distal section in a flared state is advanced in a distal direction through the introducer tool 450, its leading distal edge may catch or snag on the edges of the flushing ports 455.

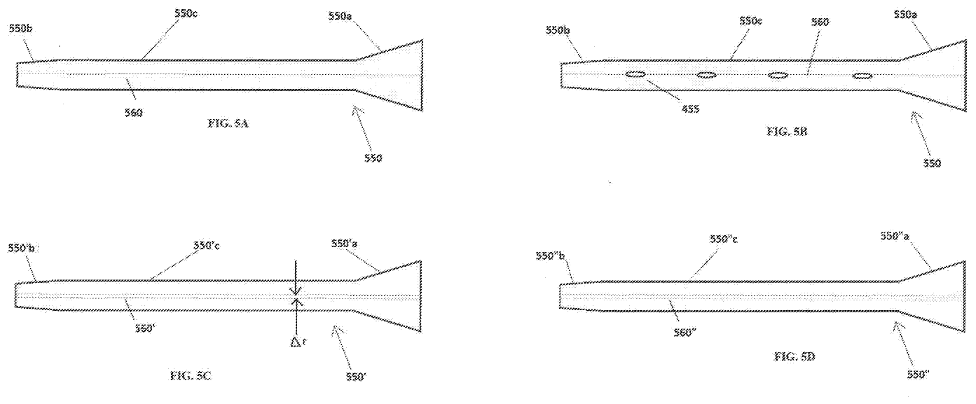

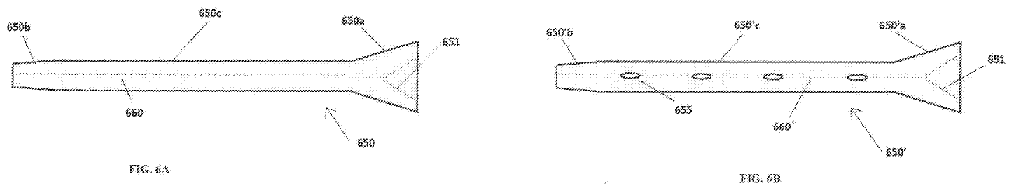

While still another alternative example of the introducer tool 550 having a single axial/longitudinal split 560 is shown in FIGS. 5A-5D. In the side view of FIG. 5A, introducer tool 550 is similar in design to that of FIG. 3A, with the only exception being the single split 560 extending in an axial/longitudinal direction from the proximal end to the opposite distal end. Introducer tool 550 separable along the split 560 so as to be easily removed from around the funnel catheter after insertion into the assembly (i.e., the hemostasis valve 105, tapered guide sheath luer/hub 120, and guide sheath catheter 125) is suitable for reuse. FIG. 5B is a side view of the introducer tool 550 similar in design to that of Figure but with the single axial/longitudinal split 560 aligned with (i.e., intersecting) flushing ports 555 (e.g., intersecting the centers of the flushing ports). The flushing ports 555 shown in the example of FIG. 5B as being aligned with the single axial/longitudinal split 560 allow entrained air to exit therethrough as the funnel catheter 175 is advanced in a distal direction through the introducer tool 550. In addition to air, fluid is also able to pass via the flushing ports 555 into the lumen introducer tool 550 by way of back pressure of blood in a proximal direction and/or positive saline flushing through the hemostasis side port 115.

FIG. 5C is yet another variation of the axial/longitudinal split design of the introducer tool 550′ similar to that of FIG. 5A. However, in FIG. 5A the two axial/longitudinal edges along the single split 560′ are in direct physical contact with each other, whereas in FIG. 5C there is a radial opening or separation Δr in the split 560′ between the complementary axial/longitudinal edges (not contacting each other—thereby having a “C” shape radial cross-section). The radial opening or separation allowing passage therethrough and into the lumen of the introducer tool 550′ of entrained air, back pressure of blood and/or positive saline flush through the hemostasis side port 115. A final variation of the split design of the introducer tool 550″ in FIG. 5D has the complementary axial/longitudinal edges along the single split 560″ radially overlapping each other. Overlapping radially of the edges along the slit 560″ prevents the risk associated with the variations of FIGS. 5A-5C of possible snagging of the flared distal end/tip of the funnel catheter along the slit 560″ when advanced through the introducer tool 550″. Despite being depicted for an introducer tool whose distal section is tapered (both in inner and outer diameters), any of the split design configurations in FIG. 5A-5D are equally suitable for an introducer tool having any alternative design, such as, but not limited to, a straight (i.e., cylindrical, uniform in both inner and outer diameter, non-tapered) distal section of FIGS. 1A-1B; tapered outer diameter distal section of FIGS. 2A & 2B; or any other design of the introducer tool illustrated and described herein.

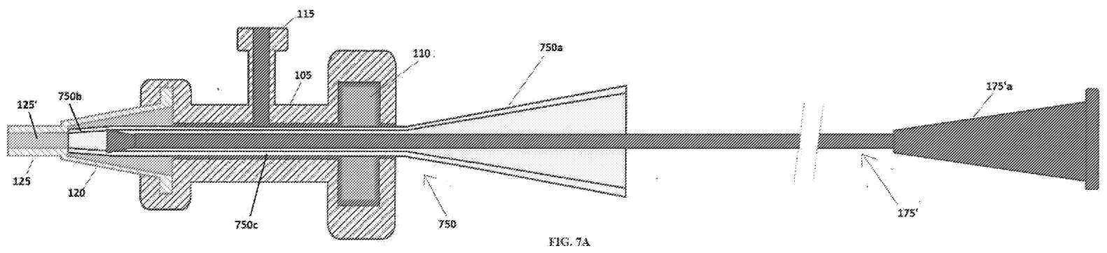

As still another alternative example, the introducer tool 650 in FIG. 6A may be peeled away in one or more axial/longitudinal section(s) without having to remove the funnel catheter from the assembly (i.e., the hemostasis valve 105, tapered guide sheath luer/hub 120, and guide sheath catheter 125). Along one or more weakened axial/longitudinal sections 660 the introducer tool 650 may be separated/torn apart for easy removal from around the funnel catheter. The weakened section(s) may be: (i) a series of perforations (as depicted in FIGS. 6A & 6B); (ii) section(s) of weaker material (e.g., an axial/longitudinal section(s) of material weaker than the material forming the remainder/rest of the tube diameter of introducer tool 650); and/or (iii) section(s) of thinner wall (e.g., an axial/longitudinal section(s) of material having a thinner wall relative to the material forming the remainder/rest of the tube diameter of the introducer tool 650). Alternatively, the introducer tool may be made from a material that has linear tearing properties where the tear is initiated by a cut at one end of the introducer (e.g., at the notch 651 and propagates axially/longitudinally. By way of illustrative example, the introducer tool 650 may include two weakened sections 660 separated radially 180° from each other allowing tearing/peeling away of two axial/longitudinal strips of the introducer tool 650 along the respective weakened sections 660. There may be more than two weakened sections radially separated from one other, equidistantly or not. It is also contemplated to have a single weakened section 660 along which when torn the introducer tool as a single sheet may be unraveled or peeled away from about the funnel catheter. The weakened section(s) 660 may be arranged in an axial/longitudinal direction or helical. An advantage associated with the peel away design is that since there is no axial/longitudinal split in the introducer tool there is no snagging of the leading distal edge of the funnel catheter when advanced in a distal direction through the introducer tool. FIG. 6B depicts an introducer tool 650′ once again peelable in an axial/longitudinal direction along one or more weakened sections 660′ aligned with a plurality of flushing ports 655 (e.g., aligned with the centers of the plurality of flushing ports). Alignment of the weakened section with some or all of the plurality of flushing ports reduces the force required to tear the introducer tool along that section, but need not necessarily be aligned with one another. As mentioned in other previous examples employing flushing ports 655, allows entrained air to exit as the funnel catheter is advanced in a distal direction. Fluid (e.g., back pressure of blood and/or positive saline flush through the hemostasis side port 115) may also pass via the flushing ports 655 into the introducer tool 650′. Despite being depicted for an introducer tool whose distal section is tapered (both in inner and outer diameters), any of the peel away design configurations (FIGS. 6A & 6B) are equally suitable for an introducer tool having a straight (i.e., cylindrical, uniform inner and outer diameters, non-tapered) distal section as in FIGS. 1A-1B; tapered outer diameter distal section of FIGS. 2A & 2B; or any other design of the introducer tool illustrated and described herein. The peel away design configurations of the introducer tool 650, 650′ in FIGS. 6A & 6B also preferably includes the notch 651 coinciding with the cone/flared proximal section 650a, 650b to facilitate the initial peel and visually indicate the region(s) to be held and pulled apart.

In the previous examples, as soon as the funnel catheter is advanced into the guide sheath catheter, the introducer tool is removed/withdrawn in a proximal direction from the hemostatic valve in order to minimize blood loss. The interventionalist typically fully removes the introducer tool from the funnel catheter at this stage also. If the working length of funnel catheter isn't a concern, the introducer tool may remain in position around the shaft of the funnel catheter and only partially withdrawn to a position proximal to the hemostatic valve, only to be later removed if, while in that position, further advancement of the funnel catheter into the assembly is hindered.

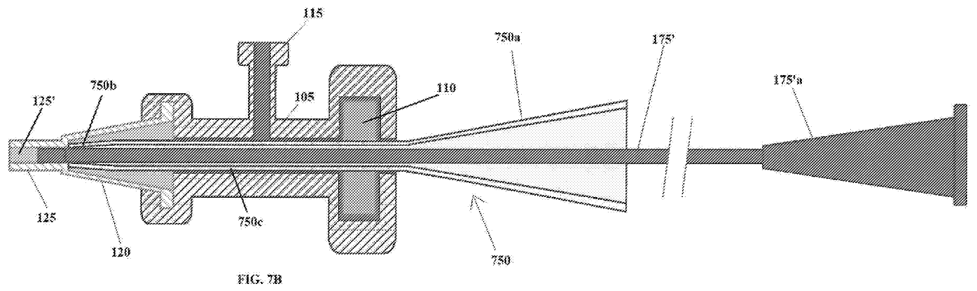

However, a modified design in FIGS. 7A-C allows the introducer tool 750 to remain in place in the assembly when the funnel catheter 175′ is fully inserted into the hemostasis valve 105 and guide sheath catheter 125. The introducer tool shown in FIGS. 7A-7C is structurally the same as that described and shown in FIGS. 3A-3D having a tapered (both in inner and outer diameters) distal section 750b with the exception of having a cone/flared proximal section 750a that is sized and shaped to fit over a cone/flared funnel catheter hub 175′a nestable therein thereby maximizing insertion of the funnel catheter 175′ into the assembly. In the example illustrated, in both size and shape the flared proximal section 750a of the introducer tool 750 conforms, matches, complements that of the flared funnel catheter hub 175′a so that the components are nestable together, one inside the other. Furthermore, the maximized axial/longitudinal length of the introducer tool 750 enhances the grip/hold thereof by the interventionalist. Still further, the enlarged flared proximal section 750a of the introducer tool 750 is prevented from being received in (i.e., entering) the lumen of the hemostasis valve 105. As previously described with respect to the earlier examples, pre-assembly (i.e., prior to insertion into the assembly (i.e., hemostasis valve 105 connected to the guide sheath catheter 125 via a tapered guide sheath luer/hub 120)) in FIG. 7A the funnel catheter 175′ is inserted into the introducer tool 750. FIGS. 7A-7C illustrate the pre-assembled introducer tool 750 and funnel catheter 175′ together as a single unit at sequential stages of insertion into the assembly. Specifically, in FIG. 7A the flared distal section of the funnel catheter 175′ is radially compressed (i.e., reduced in outer diameter) as it passes through the tapered (both in inner and outer diameters) distal section 750b of the introducer tool 750. During continued advancement in a distal direction the radially compressed distal section of the funnel catheter 175′ transfers into the lumen 125′ of the guide sheath catheter 125, as shown in FIG. 7B. Maximum or full insertion of the funnel catheter 175′ into the lumen 125′ of the guide sheath catheter 125 is depicted in FIG. 7C. The flared funnel catheter hub 175′a nested within the flared proximal section 750a of the introducer tool 750 in FIG. 7C prohibits further advancement in a distal direction into the assembly. When maximum or full insertion of the funnel catheter 175′ into the assembly is realized, the funnel catheter hub 175′a and flared proximal section 750a of the introducer tool 750 may be releasably locked or secured together using a conventional mechanical device (e.g., clip, friction tight fit, etc.). Optionally, a gasket, O-ring or other device for forming a fluid tight seal may be disposed between the introducer tool 750 and the funnel catheter 175′ to prevent blood flushing back in a proximal direction therethrough during use. Furthermore, a strain relief device may optionally be positioned about the funnel catheter 175′ distally of the funnel catheter hub 175a′ blocking/plugging the distal end of the flared proximal section 750a preventing blood loss therethrough. This adapted design of a flared proximal section 750a sized and shaped to fit over the flared funnel catheter hub 175′a to maximize insertion of the funnel catheter 175′ through the guide sheath catheter 125 and hemostasis valve 105 is suitable for use with any example of the introducer tool described herein.

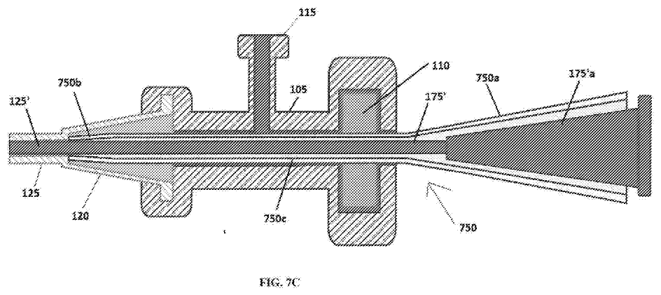

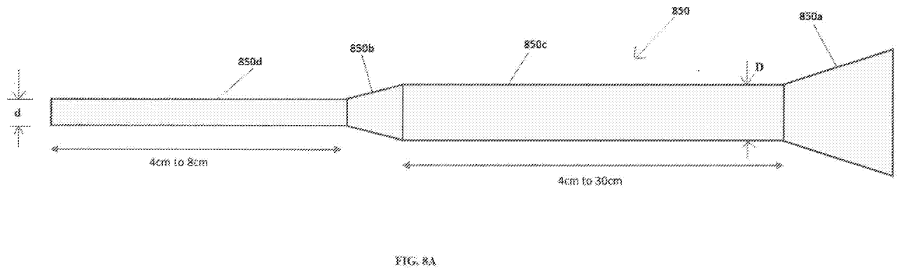

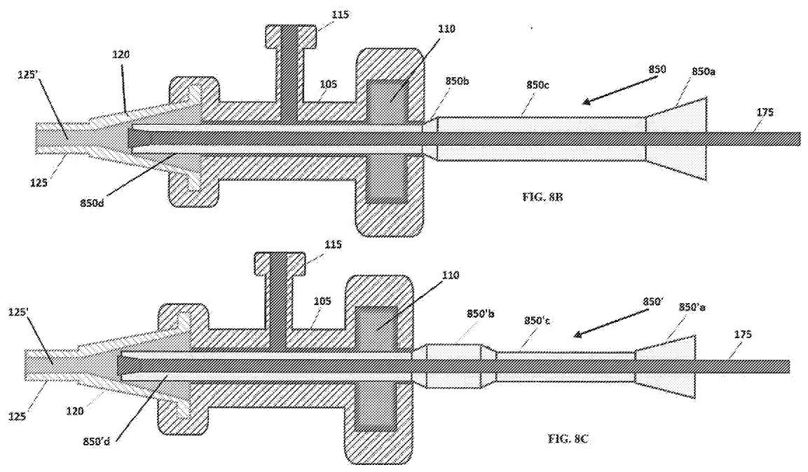

FIG. 8A depicts yet a further modification to the introducer tool addressing the particular use with a hemostasis valve whose axial/longitudinal lumen has an inner diameter so small as to be unable to receive therein the larger outer diameter of the intermediate section 350c of the introducer tool 350 of FIG. 3A. In such circumstance, the introducer tool 850 is modified to include an outer profile transition section 850b disposed between two straight (i.e., cylindrical, uniform in both inner and outer diameters) section 850c (having an outer diameter (D)) and straight section 850d (having an outer diameter (d)). The dimensions of the introducer tool 850 are selected so that the outer diameter (d) of straight (i.e., cylindrical, uniform in both inner and outer diameters) section 850d is: (i) smaller relative to that of the outer diameter (D) of the straight (i.e., cylindrical, uniform in both inner and outer diameters) section 850c; and (ii) sized to be receivable within the hemostasis valve. An outer profile of the transition section 850b advantageously limits insertion in an axial/longitudinal direction into the hemostasis valve thus preventing compression of the distal end/tip of the introducer tool in the guide catheter luer/hub avoiding constriction of the distal end/tip of the lumen of the introducer tool and excessive funnel compression. FIG. 8A depicts the outer profile transition section 850b as a tapered region, however, other alternatives are contemplated, for example, a stepped profile, ring or flange. Although the inner diameter of the introducer tool need not be constant or uniform from its proximal end to its opposite inner end, nevertheless the inner diameter of each of the flared proximal section 850a as well as the straight (i.e., cylindrical, uniform in both inner and outer diameters) section 850c of the introducer tool is able to accommodate unhindered (i.e., free from radial compression) the distal section while in a flared (i.e., open biased, radially enlarged, non—compressed radially of maximum outer diameter) state of the funnel catheter 175.

As with the previously described examples, once again the funnel catheter 175 with the distal section in a flared (open biased, radially enlarged, non-compressed radially of maximum outer diameter) state is pre-assembled in the introducer tool 850 of FIG. 8A and the two components together as a single unit is advanced through the assembly (i.e., the hemostasis valve 105 connected to the guide sheath catheter 125 via the tapered guide sheath lure/hub 120), as illustrated in FIG. 8B. Only the straight (i.e., cylindrical, uniform in both inner and outer diameters) extension section 850d is sized to be received within the axial/longitudinal lumen of the hemostasis valve 105, whereas the outer profile transition section 850b, straight (i.e., cylindrical, uniform in both inner and outer diameters) section 850c and flared proximal section 850a too large in diameter preventing from being received within remain proximally (outside) of the hemostasis valve 105. With continued advancement (i.e., pushing in a distal direction) through the introducer tool 850, upon emerging out from the distal end/tip thereof, the flared (open biased) distal section of the funnel catheter 175 is radially compressed by the tapered inner wall of the tapered guide sheath catheter 125. In FIG. 8A the length in an axial/longitudinal direction of the straight (i.e., cylindrical, uniform in both inner and outer diameters) section 850c (i.e., from a distal end/tip of the flared proximal section 850a to a proximal end/tip of the outer profile transition section 850b) ranges between approximately 4 cm—approximately 30 cm. When inserted into the flared proximal section 850a and advanced in a distal direction through the straight (i.e., cylindrical, uniform in both inner and outer diameters) section 850c of the introducer tool 850 the distal section of the funnel catheter 175 is maintained in a flared (i.e., open biased, radially enlarged, non-compressed radially of maximum outer diameter) state.

As yet another alternative design to limit insertion depth in the assembly and prevent compression of the distal end/tip, both the inner and the outer profiles of the shaft of the introducer tool may be a continuous taper to the flared proximal section 850a′, as depicted in FIG. 8F.

In FIG. 8A support of the funnel catheter shaft within the straight (i.e., cylindrical, uniform in both inner and outer diameters) section 850c of the introducer tool 850 is poor due to the space or gap therebetween. To enhance or improve support of the funnel catheter shaft (i.e., reduce the space or gap between the straight (i.e., cylindrical, uniform in both inner and outer diameters) section 850′c and the funnel catheter shaft), the difference in the outer diameter of the straight (i.e., cylindrical, uniform in both inner and outer diameters) section 850′c and the outer diameter of the straight (i.e., cylindrical, uniform in both inner and outer diameters) extension section 850′d is minimized, preferably equal, connected via an outer profile transition section 850′b that transitions bi-directionally (i.e., transitions both in the proximal direction and the distal direction). Referring to FIG. 8C, the outer diameter of the straight ((i.e., cylindrical, uniform in both inner and outer diameters) section 850′c and the outer diameter of the straight ((i.e., cylindrical, uniform in both inner and outer diameters) extension section 850′d are equal. The outer profile transition section 850′b transitions bi-directionally (i.e., transitions both in the proximal direction and the distal direction) with an intermediate straight (i.e., cylindrical, uniform in both inner and outer diameters) section between opposing transitions. An external flange with a continuous inner diameter may be substituted for the intermediate straight (i.e., cylindrical, uniform in both inner and outer diameters) section 850′b between opposing transitions. Other variations of the outer profile transition section 850′b are contemplated so long as the configuration limits the depth of insertion of the introducer tool into the assembly.

Alternatively, the intermediate straight (i.e., cylindrical, uniform in both inner and outer diameters) section may be omitted whereby the opposing transitions abut each other, which in a longitudinal cross-section forms a diamond shape. Once again, the tapered transition in opposing directions (e.g., tapering to a smaller diameter in a proximal direction and tapering to a larger diameter in a distal direction) may be replaced by a stepped transition. The multi-diameter section introducer tool 850′ in FIG. 8C is sized so that when fully inserted into the hemostasis valve 105 the following conditions are satisfied: (i) the bi-directional outer profile transition section 850b′, straight (i.e., cylindrical, uniform in both inner and outer diameters) section 850′c and flared proximal section 850′a remain outside (proximal) of the hemostasis valve 105; and (ii) preventing compression of the distal end/tip and narrowing of the lumen of the introducer tool 850′ by avoiding radial interference with the tapered inner wall/surface of the guide catheter luer/hub 120.

In FIG. 8B the sizing of the outer profile transition section 850b and straight (i.e., cylindrical, uniform in both inner and outer diameters) section 850c is selected to be greater than the inner diameter of the lumen of the hemostasis valve, thereby preventing insertion of theses sections therein (i.e., only the straight (i.e., cylindrical, uniform in both inner and outer diameters) distal extension section 850d being sized to be received in the lumen of the hemostasis valve). By varying the size of the respective sections of this same construction or design of the multi-diameter introducer tool of FIG. 8A the way it engages with the assembly (i.e., the hemostasis valve connected to the guide sheath catheter via the guide sheath luer/hub) may be modified. Referring to FIG. 8D, the outer diameter of the straight (i.e., cylindrical, uniform in both inner and outer diameters) section 850c may be sized to be smaller than the inner diameter of the lumen of the hemostasis valve 105 to be accommodated (insertable therein) along with the outer profile transition section 850b (e.g., transitioning unidirectionally or bi-directionally) and distal extension section 850d. The distal extension section 850d has a uniform outer diameter that is substantially equal (preferably equal) to the inner diameter of the lumen of the guide sheath catheter 125. The distal section of the funnel catheter while in a flared (i.e., open biased, radially enlarged, non-compressed radially of a maximum outer diameter) state is accommodated unhindered (i.e., free from radial compression) while traversing in a distal direction through the flared proximal section 850a and the straight (i.e., cylindrical, uniform in both inner and outer diameters) section 850c. With continued advancement of the funnel catheter through the assembly, the flared distal section is radially compressed (i.e., reduced in outer diameter) as it travels through the outer profile transition section 850b and distal extension section 850d, wherein sections 850b, 850d each have an inner diameter smaller than the maximum outer diameter of the funnel in a flared open biased state. In an axial/longitudinal direction a length of the distal extension section 850d is preferably minimized thereby minimizing a distance traversed in an axial/longitudinal direction of the funnel catheter while its distal section is in a radially compressed state (i.e., reduced in outer diameter). A proximal region X of the funnel catheter 175 is preferably made of a material stiffer (i.e., less flexible) relative to the remaining region distally thereof that is made of a more flexible (i.e., less stiff) material. The overall axial/longitudinal length (including the proximal section 850a, straight (i.e., cylindrical, uniform in both inner and outer diameters) section 850c, and outer profile transition section 850b) of the introducer tool 850 in FIG. 8D is in a range of approximately 4 cm—approximately 8 cm. Accordingly, when the introducer tool 850 is fully inserted into the assembly with its distal end/tip in direct physical contact with the tapered inner wall/profile of the tapered guide sheath luer/hub 120, a portion of the funnel catheter made of a material more flexible and arranged distally of the stiffer proximal region is undesirably unsupported by the introducer tool. FIG. 8E depicts an introducer tool similar to that of FIG. 8D but longer in overall axial/longitudinal length (including the flared proximal section 850a, straight (i.e., cylindrical, uniform in both inner and outer diameters) section 850c, and outer profile transition section 850b) that is in a range of approximately 20 cm—approximately 30 cm. Increasing the overall length of the introducer tool advantageously maximizes support provided to the flexible flared distal section of the funnel catheter 175″ while allowing the interventionalist to grip the stiffer proximal region X of the introducer tool.

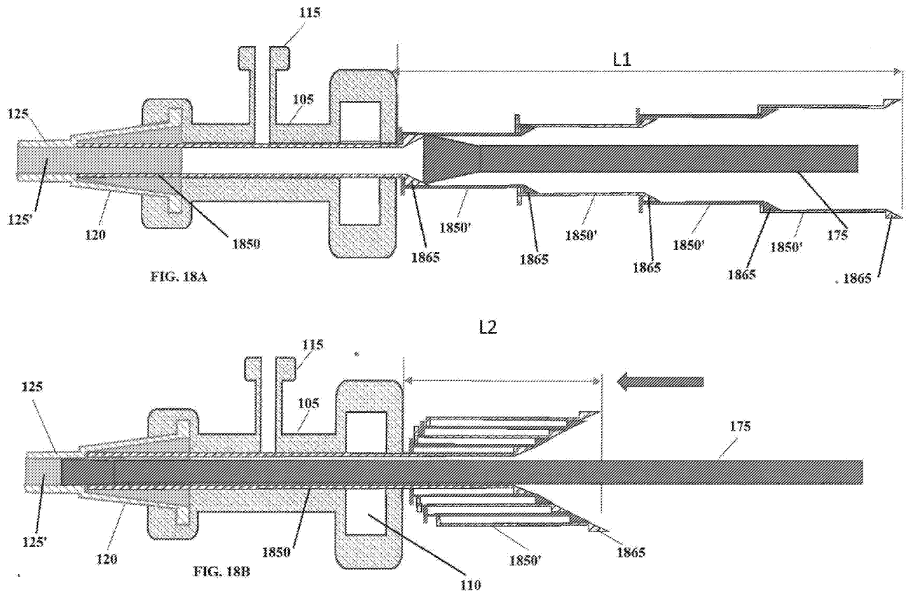

As previously mentioned, it is desirable to maximize the working length in an axial/longitudinal direction of the introducer tool through which the funnel catheter passes. This may be achieved, by designing the introducer tool to include an axially/longitudinally non-contractable section 1850 and proximal thereto an axially/longitudinally contractable section of telescopic concentric tubular sections 1850′ designed to slide into one another. In a contracted state, the telescopic concentric tubular sections 1850′ are too large to be received/inserted in the hemostasis valve. In FIGS. 18A & 18B the telescopic concentric tubular sections 1850′ are depicted in an axially/longitudinally expanded (i.e., non-contracted, maximum axial/longitudinal length L1) state and an axially/longitudinally contracted (i.e., minimum axial/longitudinal length L2) state, respectively. Any number of telescopic concentric tubular sections 1850′ may be selected to achieve a desired working length with the concentric tubular sections arranged decreasing in inner diameter from its proximal end to its opposite distal end. In the example depicted in FIG. 18A the distal end of each tubular section has a stop feature (e.g., a lip) while the proximal end of each tubular section has a lead-in flange 1865 with an external square step. The lead-in flange external surface engages the stop on the tubular section proximal limiting telescopic expansion while at the same time providing a smooth internal surface for advancement of the flared distal section of the funnel catheter therethrough. Other mechanical arrangements for limiting expansion of the tubular sections are within the scope of the disclosure. While its distal section in a flared state (i.e., open biased, maximum outer diameter, radially non-compressed) the funnel catheter is introduced unhindered (i.e., free from radial compression) into the proximal end. As the funnel catheter is advanced through the telescopic concentric tubular sections 1850′ upon engaging with the integral stop and lead-in 1865 the flared distal section is radially compressed. Emerging from the distal end of the axially/longitudinally non-contractable section 1850 of the introducer tool the outer diameter of the distal section of the funnel catheter is sufficiently reduced in outer diameter to transfer as is into the lumen of the guide sheath catheter 125 without engaging the tapered inner wall of the tapered guide sheath luer/hub 120.

The telescopic concentric tubular sections in an axially/longitudinally expanded (non-contracted) state maximize the axial/longitudinal working length of the introducer tool through which the funnel catheter travels. This maximum working length is realized, while still allowing the telescopic concentric tubular sections 1850′ when axially/longitudinally contracted (i.e., collapsed to a minimum axial/longitudinal length L2) to nevertheless remain in position on/about/around the funnel catheter. The axially/longitudinally non-contractable section 1850 of the introducer tool may either remain in the hemostasis valve 105 (as depicted in FIG. 18B), or be fully withdrawn (pulled out) from the hemostasis valve 105 to avoid leakage of blood through the lumen of the introducer tool.

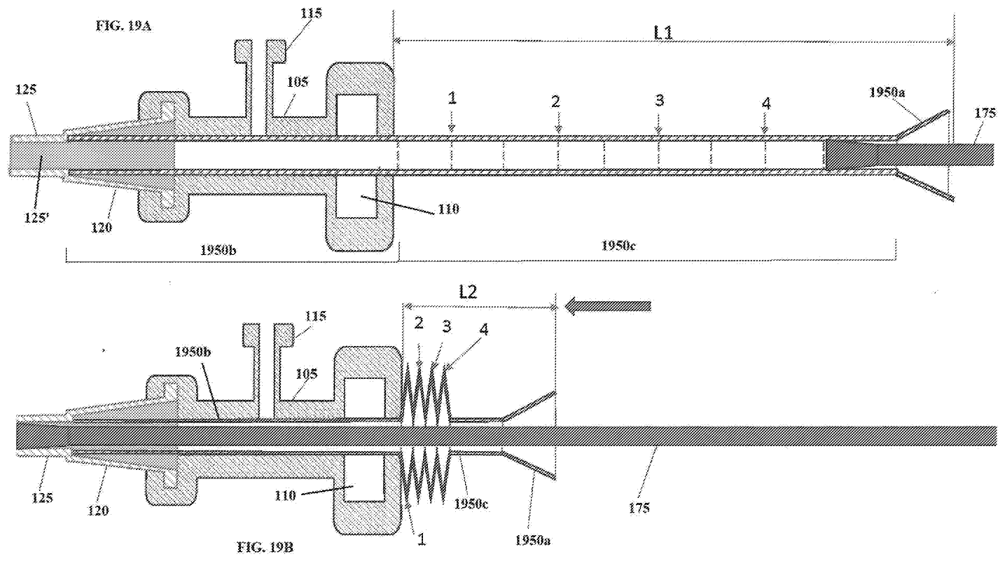

FIGS. 19A & 19B represent an alternative of the introducer tool that on the one hand maximizes the working length through which the funnel catheter traverses, while on the other hand is axially/longitudinally contractable in length. In this alternative design, the present inventive introducer tool has a cone/flared proximal section 1950a integral with a shaft section disposed distally thereof, wherein the shaft section includes an axially/longitudinally contractable section 1950c integral with an axially/longitudinally non-contractable section 1950b. The axially/longitudinally contractable section 1950c comprises a plurality of bellows (1, 2, 3, 4) transitionable from an expanded state to a contracted state, wherein the number and arrangement of bellows may be selected, as desired.

When the axially/longitudinally contractable section 1950c is in an expanded state (FIG. 19A), sections 1950b, 1950c are uniform in both inner and outer diameters. The uniform outer diameter of the non-contractable section 1950b is insertable into the hemostasis valve 105. With its distal section in a flared (i.e., open biased, radially non-compressed, maximum outer diameter) state the funnel catheter is introduced into the proximal section 1950a of the introducer tool. Upon entering the shaft section (1950c, 1950b) of the introducer tool the flared distal section of the funnel catheter is radially compressed (reduced in outer diameter) preferably substantially equal to the inner diameter of the lumen of the guide sheath catheter 125 into which the funnel catheter transfers. FIG. 19B depicts the contractable section 1950c of the introducer tool in an axially/longitudinally contracted state (i.e., collapsed) with the radially expanded (i.e., maximum outer diameter) bellows (1, 2, 3, 4) disposed proximally (i.e., outside) of the hemostasis valve 105. Once again, the maximum working length of the introducer tool is realized when the axially/longitudinally contractable section 1950c is in an expanded state, while still allowing the bellows (1, 2, 3, 4) when axially/longitudinally contracted (i.e., collapsed to a minimum axial/longitudinal length L2) to remain on/about/around the funnel catheter. The non-contractable section 1950b of the introducer tool may either remain in the hemostasis valve 105 (as depicted in FIG. 19B), or be fully withdrawn (pulled out) from the hemostasis valve 105 to avoid leakage of blood through the lumen of the introducer tool.

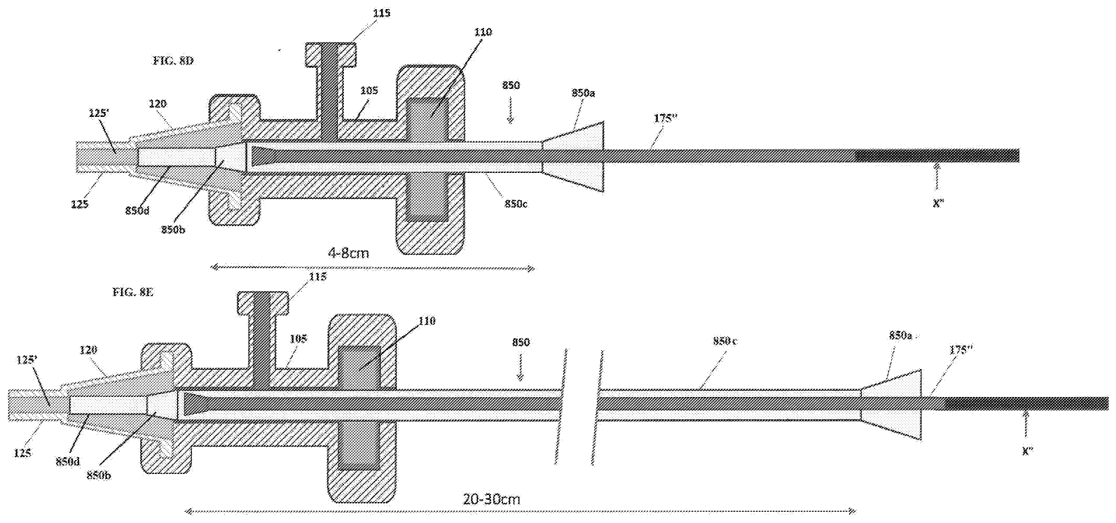

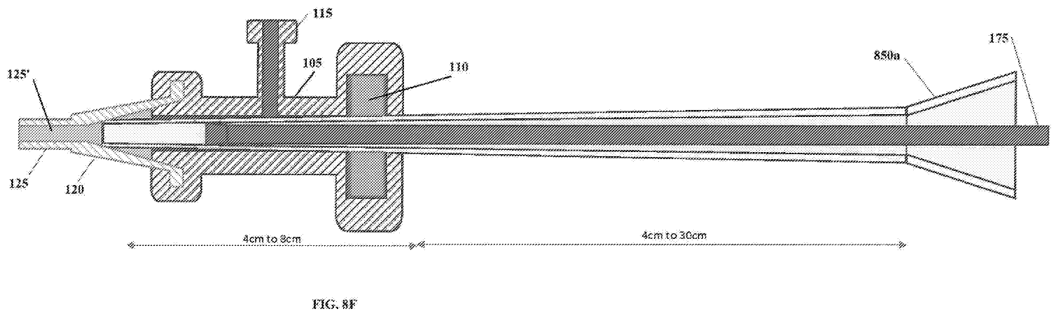

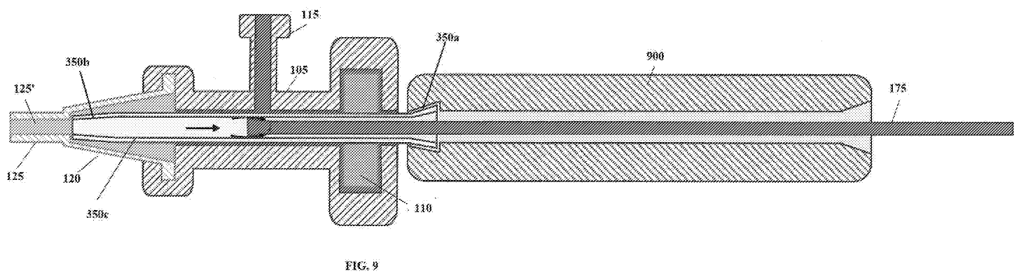

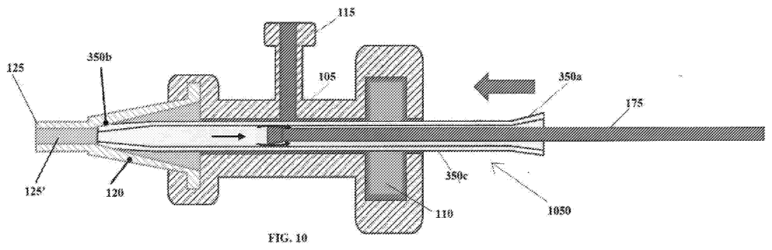

In any of the examples illustrated and described above, if subject to application of an excessive force (i.e., over insertion) in a distal direction (as denoted by the arrow in the example of FIG. 10) the distal end/tip of the introducer tool 1050 upon directly physically contacting/engaging with the inner wall/profile of the guide sheath luer/hub 120 may undesirably compress axially and/or radially (i.e., narrowing the inner diameter of the lumen) thereby restricting or preventing passage therethrough the flared distal section of the funnel catheter 175. Regardless of the imposition of an excessive force imposed on the introducer tool, it would be desirable to limit the extent/depth in an axial/longitudinal direction to which the introducer tool is advanceable through the assembly in order to prevent over insertion (i.e., compression axially and/or radially of the distal end/tip of the introducer tool against the tapered inner wall/profile of the tapered guide sheath hub/luer). Compression of the distal end/tip is prevented by designing a section of the introducer section that extends proximal (outward) of the hemostasis valve when fully inserted therein to include a radially expandable compressive force absorption component. Numerous mechanical structures associated with the introducer tool for absorbing imposition of an excessive compressive force applied in an axial/longitudinal direction are contemplated a few illustrative, but non-limiting examples, of which are described herein.

FIGS. 11A-1D show a first exemplary introducer tool 1150 including a radially expandable compressive force absorption component 1195 configured as a series of radial pleats, folds or radially corrugated (i.e., alternating radial peaks and radially valleys) resembling that of an accordion. The number and spacing of the pleats may be modified to absorb a desired maximum axial/longitudinal compressive force. Starting from its proximal end/tip, the introducer tool 1150 in FIGS. 11A-11D includes a handle 1150d and distally thereof a non-insertable section 1150a followed thereafter by an insertable straight (i.e., cylindrical, uniform in both inner and outer diameters) extension section 1150c with a transition section 1150b (e.g., tapered or stepped) disposed between the sections 1150a, 1150c. The insertable straight (i.e., cylindrical, uniform in both inner and outer diameters) extension section 1150c has an outer diameter/outer profile sized to be receivable in the lumen of the hemostasis valve 105, whereas the non-insertable section 1150a has an associated outer diameter/outer profile greater than (i.e., not receivable/insertable in) the lumen of the hemostasis valve 105. Thus, the outer diameter/outer profile of the non-insertable section 1150a is greater than the outer diameter/outer profile of the insertable straight (i.e., cylindrical, uniform in both inner and outer diameters) extension section 1150c.

FIGS. 11A & 11C show the introducer tool 1150 itself with the radially expandable compressive force absorption component (i.e., pleats) 1195 depicted in axially/longitudinally non-compressed (expanded) and compressed states, respectively. These respective non-compressed (expanded) and compressed states of the radially expandable compressive force absorption component (i.e., pleats) 1195 of the introducer tool 1150 in use while inserted into the assembly (i.e., the hemostasis valve 105 connected to the guide sheath catheter 125 via the tapered guide sheath catheter luer/hub 120) are depicted in FIGS. 11B & 11D, respectively. The funnel catheter 175 with its distal section in a flared (i.e., open biased, radially enlarged, non-compressed radially of maximum outer diameter/profile) state is advanceable unhindered through the lumen of the introducer tool 1150 while the radially expandable compressive force absorption component 1195 is in a non-compressed (expanded) state. Outside of the body prior to insertion into the assembly, the funnel catheter 175 whose distal section is in a flared (i.e., open biased, radially enlarged, non-compressed radially of maximum outer diameter) state is pre-assembled in the introducer tool 1150 (while the radially expandable compressive force absorption component 1195 is in a non-compressed (expanded) state). The introducer tool 1150 and funnel catheter 175 together as a single unit is advanced in a distal direction through the assembly (FIG. 11B). At least a portion, possibly all, of the transition section 1150b remains proximal (outside) of the hemostasis valve 105. Referring to FIG. 11B, prior to application of an excessive axial/longitudinal compressive force on the introducer tool 1150, the series of radial pleats comprising the radially expandable compressive force absorption component 1195 are axially/longitudinally expanded (i.e., axially/longitudinally non-compressed (expanded) with a maximum distance separation in an axial/longitudinal direction between adjacent radial peaks). When the introducer tool 1150 is subject to over insertion exceeding that for full insertion without undesirably compressing the distal end/tip, as illustrated in FIG. 11D, the series of pleats comprising the radially expandable compressive force absorption component 1195 compresses in an axial/longitudinal direction (i.e., reduces the axial/longitudinal separation between adjacent radial peaks) absorbing the excess load thwarting unwanted compression of the distal end/tip.

An alternative of the present inventive introducer tool 1250 includes a radially expandable compressive force absorption component 1295 comprising a plurality of radially outward bendable arms disposed between a series of slits defined in the non-insertable section 1250a with the slits radially separated from one another and extending in an axial/longitudinal direction parallel to each other, in FIGS. 12A-12D. The number of slits, radial width of each slit, axial/longitudinal length of each slit, and/or radial separation between adjacent slits may be modified, as desired, to absorb a desired maximum compression load in an axial/longitudinal direction. The slits reduce the rigidity of the non-insertable section 1250a establishing a series of weakened axial sections in those remaining areas therebetween. Stiffness of the remaining weakened sections between adjacent slits and thus a maximum compressive force able to be absorbed may be varied, as desired, based on selection of the number of slits, axial/longitudinal length of each slit, width in a radial direction of each slit, and/or radial separation between adjacent slits. When the introducer tool is subject to excessive compressive load in an axial/longitudinal direction, the weakened remaining axial sections (i.e., arms) expand radially outward (the presence of the flared distal section of the funnel catheter 175 within the internal lumen of the introducer tool 1250 preventing radially inward compression). In comparison with the accordion design of FIGS. 11A-11D in which adjacent peaks 1195 are separated from one another in an axial/longitudinal direction, in the alternative design of FIGS. 12A-12D, adjacent radially outward bent arms are separated radially from one other. FIGS. 12A & 12C depict the introducer tool 1250 including radially outward bendable arms 1295 defined by a series of axial/longitudinally defined slits in the non-insertable section 1250a, wherein the weakened remaining sections (i.e., arms) are not subject to an excessive compressive load (FIG. 12A) and subject to an excessive compressive load (FIG. 12C). The maximum outer diameter/outer profile of the radially outward bent arms 1295 occurring while subject to an excessive compression load (FIG. 12C), whereas when not subject to excessive compressive load the radially outward bendable arms 1295 have a minimum outer diameter/outer profile (preferably equal in outer diameter/profile to the outer diameter/profile of the remaining non-insertable straight (cylindrical) section 1250a of the introducer tool that does not bend radially outward when subject to an excessive compressive force)(FIG. 12D). These respective non-compressed and compressed states of the introducer tool 1250 in use while inserted into the assembly (i.e., the hemostasis valve 105 connected to the guide sheath catheter 125 via a tapered guide sheath luer/hub 120) are depicted in FIGS. 12B & 12D, respectively. Once again, the funnel catheter 175 while its distal section is in a flared (i.e., open biased, radially enlarged, non-compressed of maximum outer diameter) state is advanced unhindered through the lumen of the introducer tool 1250 while the radially expandable compression force absorption component 1295 is in a non-compressed state (i.e., minimum outer diameter/profile). Together as a single unit the introducer tool 1250 with the radially expandable compression force absorption component 1295 in a non-compressed state along with the funnel catheter 175 inserted therein with its distal section in a flared (i.e., open biased) state is advanced in a distal direction through the assembly. The outer diameter/outer profile along at least some portion of the transition section 1250b remaining outside of the hemostasis valve. Referring to FIG. 12B, prior to application of an excessive axial/longitudinal compressive load (i.e., excessive pushing in a distal direction) on the introducer tool 1250, the radially expandable compression force absorption component (i.e., radially outward bendable arms) 1295 disposed between the adjacent slits have a minimum outer diameter. When the introducer tool 1295 is subject to over insertion exceeding that for full insertion without compression of the distal end/tip, as illustrated in FIG. 12D, the weakened remaining sections between adjacent slits flare (e.g., bend) forming radially outward bent arms 1295 having a maximum outer diameter/profile. These flared radially outward bendable arms 1295 absorb the excessive compressive load thereby thwarting unwanted compression of the distal end/tip of the introducer tool.

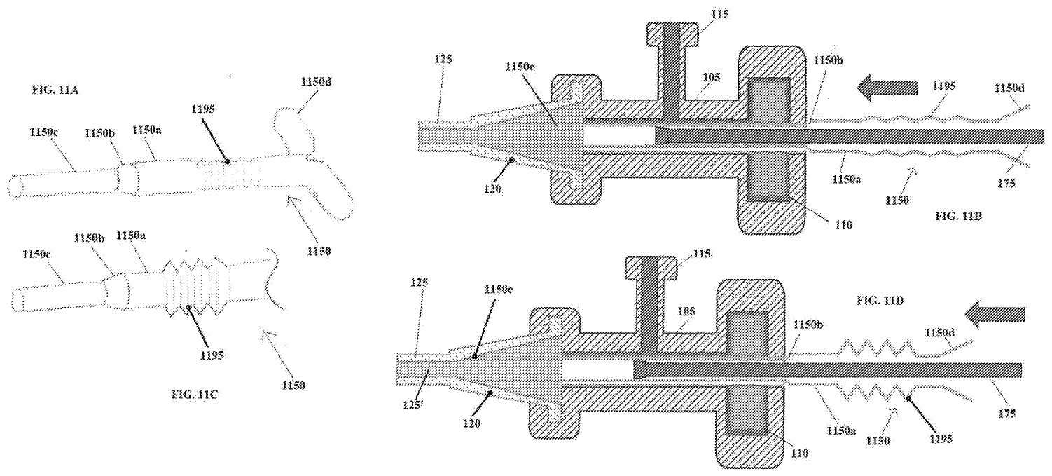

The slits forming the radially outward bendable arms 1295 of the introducer tool 1250 (FIGS. 12A-12D) are arranged parallel to one another in an axial/longitudinal axis of the introducer tool. However, the slits, still parallel to one another, may alternatively be arranged oblique (i.e., non-parallel and non-perpendicular to the axial/longitudinal axis through the introducer tool 1350), as depicted in FIGS. 13A-13D. Similar to that of FIGS. 12A-12D the weakened remaining sections between adjacent slits flare (e.g., bend) forming radially outward bent arms 1395 thereby absorbing the compressive load preventing compression of the distal end/tip of the introducer tool. Aside from the oblique arrangement of the slits in FIGS. 13A-13D relative to the arrangement parallel to the axial/longitudinal axis through the introducer tool in all other respects the disclosure of one introducer tool is applicable to that of the other.

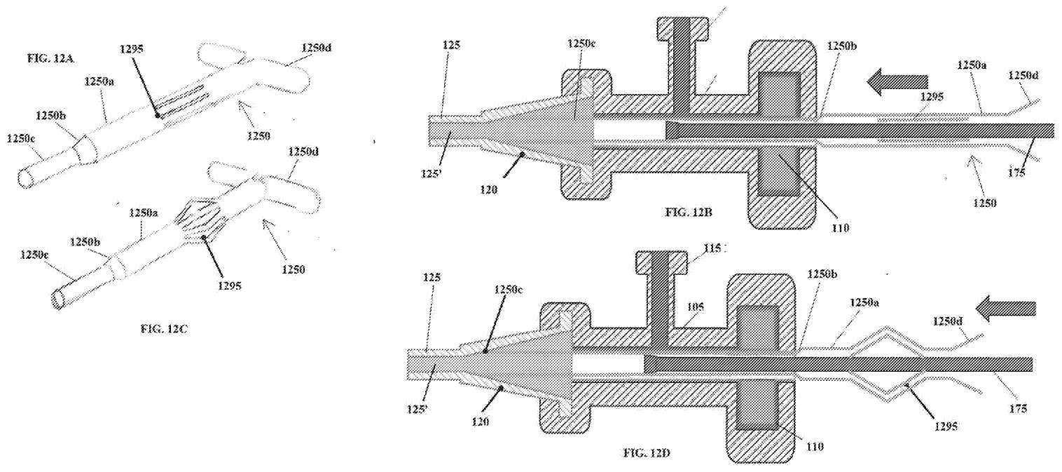

It is further contemplated to stagger in a radial direction and/or an axial/longitudinal direction the slits defined in the non-insertable section 1450a of the introducer tool providing a radially expandable compression force absorption component 1495 that bulges/expands when subject to a compressive force. In FIGS. 14A-14D, at the same radial position a first series of axial/longitudinal slits are defined parallel to the axial/longitudinal axis of the introducer tool 1450. A next adjacent series of axial/longitudinal slits are arranged parallel, separated radially from and offset in an axial/longitudinal direction relative to the first series of axial/longitudinal slits. It is worth noting, that the series of slits radially separated from one another need not necessarily be offset in an axial/longitudinal direction relative to each other but could otherwise be aligned. FIGS. 14A & 14C depict the introducer tool 1450 depicting the radially expandable compression force absorption component 1495 in a non-compressed state and a compressed state, respectively. Similarly, FIGS. 14B & 14D depict the introducer tool 1450 of FIGS. 14A & 14C together as a single unit with the funnel catheter inserted into the assembly depicting the radially expandable compression force absorption component 1495 in the non-compressed state and compressed state, respectively.

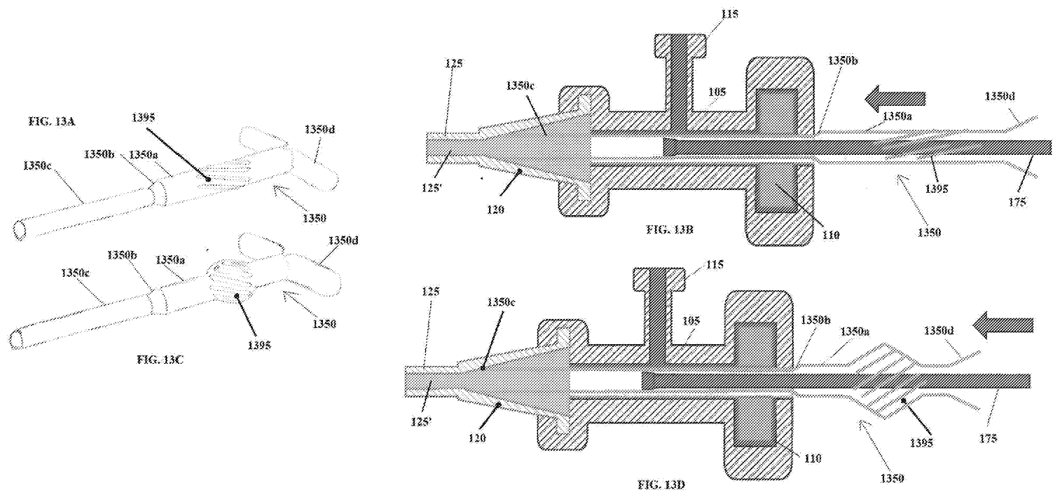

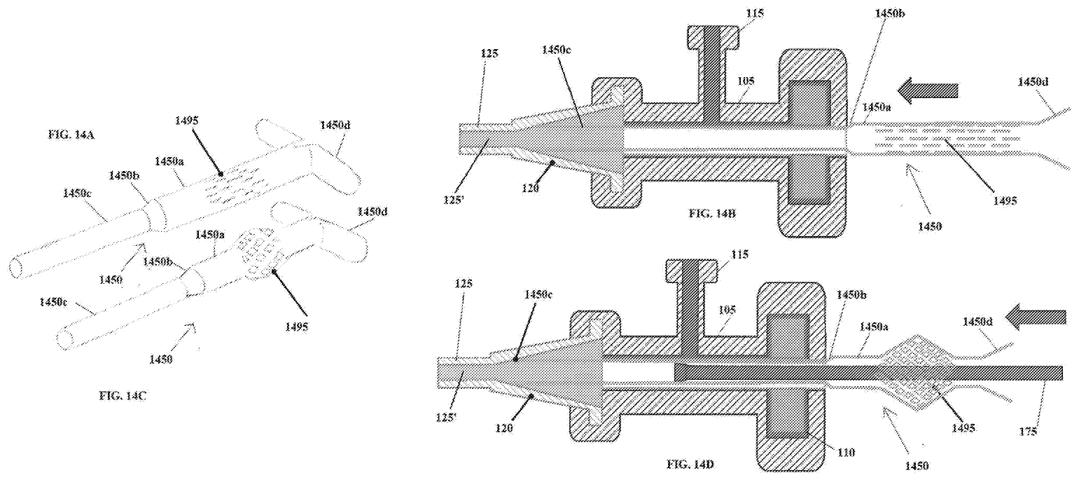

With the previously described introducer tools employing a radially expandable compression force component, the material of the introducer tool forming the radially expandable compression force absorption component is the same as the remaining section comprising the non-insertable section 1550a. As yet another possible alternative to the creation of pleats or slits, the material used for the radially expandable compression force component may differ in stiffness. Specifically, the radially expandable compression force absorption component 1595 of the introducer tool 1550 may be made of a softer/lower stiffness/more flexible material (preferably in a range approximately 10D-approximately 40D or approximately 30A-approximately 80A) relative to that of the harder/more stiff/less flexible material (preferably in a range approximately 40D-approximately 80D) of the remaining sections comprising the non-insertable section 1550a of the introducer tool. That is, a lower durometer material is used for the radially expandable compression force absorption component 1595 relative to that for the remaining sections comprising the insertable straight (i.e., cylindrical, uniform in both inner and outer diameters) section 1550a of the introducer tool. Again, the funnel catheter 175 with its distal section in a flared (i.e., open biased, maximum outer diameter/profile) inserted unhindered into the introducer tool while the radially expandable compression force absorption component free from application of a compressive force has a minimum outer diameter/profile (preferably uniform and equal in outer diameter/profile to the rest of the non-insertable straight (i.e., cylindrical, uniform in both inner and outer diameters) section 1550a of the introducer tool (FIG. 15A)). When subject to a compressive force, the less stiff material comprising the radially expandable compression force absorption component 1595 bulges radially outward thereby absorbing any compressive force due to over insertion of the introducer tool into the assembly (FIG. 15C). In use, together as a single unit the introducer tool (while the minimum outer diameter/profile of the radially expandable compression force absorption component is maintained) and the flared funnel catheter therein are advanced in a distal direction through the assembly (FIG. 15B). When the fully advanced into the assembly (i.e., the distal end/tip is proximate to without physically contacting the tapered inner wall of the tapered guide sheath luer/hub) application of additional force in a distal direction (i.e., over insertion) causes the lower stiffness material of the radially expandable compression force absorption component 1595 to bulge (i.e., expand radially outward) thereby absorbing the excessive compressive load preventing transfer to the distal end/tip of the introducer tool thus thwarting compression/narrowing/collapse of the lumen (FIG. 15D). Once again, variation in the amount of compressive force able to be absorbed may vary, as desired, by selecting one or more of the following parameters of the radially expandable compression force absorption component: (i) the material based on its stiffness; (ii) the length in an axial/longitudinal direction; and/or (iii) the thickness.

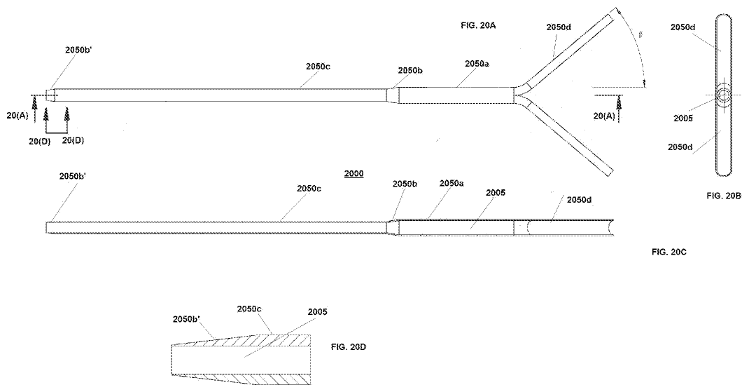

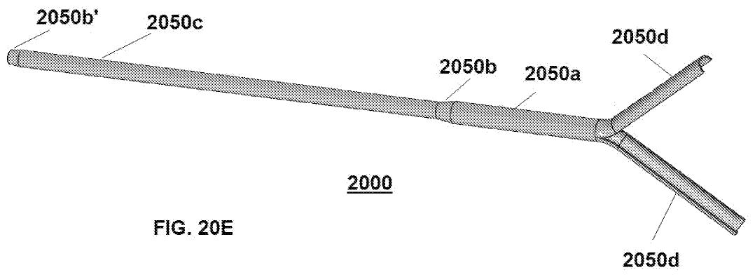

FIGS. 20A-20E depict yet another example of the introducer tool 2000 in accordance with the present disclosure. A proximal section (including the proximal end/tip) of the introducer tool 2000 is radially divided or split (e.g., peelable apart/separable along a perforation or propagation of tearing along the polymer chains of a material) into multiple sections (preferably bisected into two 180° sections) wherein each divided proximal section is radially separated/bent at an angle β relative to the axial/longitudinal axis through the introducer tool and may be flared outward from one another to form a pair of handles/tabs 2050d. Advancing in a distal direction, the next section of the introducer tool 2000 following the curved (bent) interface at the distal end of the divided proximal section 2050d is a first straight section 2050a (i.e., cylindrical, uniform in both inner diameter and outer diameter). Distally of the first straight section 2050a is a transition section 2050b followed by a second straight section 2050c (i.e., cylindrical, uniform in both inner diameter and outer diameter). Transition section 2050b tapers in diameter (both inner diameter and outer diameter) from that of the larger diameter (both inner diameter and outer diameter) of the first straight section 2050a to the smaller diameter (both inner diameter and outer diameter) of the second straight section 2050c. A distal tapered section 2050b′ (including the distal tip) of the introducer tool 2000 is tapered in outer diameter while maintaining a uniform (i.e., non-tapered, straight) inner diameter, as depicted in the enlarged partial axial/longitudinal cross-sectional view of FIG. 20D. Distally of the handles 2050d, a lumen 2005 (shown in FIG. 20B) defined in the introducer tool 2000 extends axially/longitudinally through the first straight section 2050a, the transition section 2050b, the second straight section 2050c and the tapered distal section 2050b′. A distal face of tapered distal section 2050b′ of the introducer tool is preferably blunt (e.g., rounded) providing a gentle or smooth interface with the conformable flared distal section of the funnel catheter without scrapping off the hydrophilic coating when retracted proximally therethrough. For instance, repositioning (e.g., retraction in a proximal direction) of the funnel catheter may occur if the interventionalist inadvertently advances in a distal direction too far the flared distal section of the funnel catheter. Use a transparent or translucent material for the introducer tool is advantageous so that the flared distal section of the funnel catheter remains visible relative to the distal end/tip of the introducer during assembly of the components prior to insertion of the assembled components into the hemostasis valve and subsequent advancement of the flared distal section of the funnel catheter into the guide catheter. Particular use of a colored transparent or translucent material also allows the introducer tool to be readily located while resting on a surface (e.g., surgical drapes).

By way of illustrative example, in FIG. 20A, the axial/longitudinal length of the divided proximal section 2050d of the introducer tool 2000 forming the handles is approximately 30 mm±approximately 5 mm, while the angle β of radial spreading apart/bending of each divided proximal section relative to the axial/longitudinal axis through the introducer tool 2000 is approximately 40° for handle. Advancing in a distal direction, the axial/longitudinal length of the remaining sections include: minimum of approximately 25 mm (sufficient space to hold the device between the finger and thumb) the length of first straight section 2050a; approximately 2 mm to approximately 10 mm is the length of transition section 2050b; approximately 80 mm is the length of second straight section 2050c (compatible with most readily available conventional hemostasis valves, but may be made shorter or longer); and approximately 1 mm to approximately 3 mm is the length of tapered distal section 2050b′. Continuing with the same illustrative example, the inner diameter of each of the respective sections is as follows: the first straight section 2050a has an inner diameter ≥ approximately with a wall thickness approximately 0.010″ (allowing the flared distal section of the funnel catheter having an outer diameter of approximately 0.110″ with sufficient clearance to permit passage therethrough), while the outer diameter of the first straight section 2050a of approximately 0.130″ is larger than the lumen of readily available conventional hemostasis valves having an inner diameter of approximately 0.120″ preventing over insertion of the introducer tool); the second straight section 2050c has an inner diameter of approximately 0.092″±approximately 0.002″ with a wall thickness of approximately 0.011″+/−approximately 0.002″ (the inner diameter of second straight section 2050c is close to the outer diameter of compatible conventional guide catheters ranging from approximately 0.085″ to approximately 0.095″) and an outer diameter of the second straight section 2050c is <approximately 0.0118″ (less than the inner diameter of approximately 0.120″ of readily available conventional hemo stasis valves); and at the distal tip/end of the distal tapered section 2050b′ the radial thickness is approximately 0.001″ (so that the outer diameter at the distal tip is as close as possible to the inner diameter of the guide sheath catheter) or with a rounded thickness of approximately 0.004″ (to protect hydrophilic coating on the flared tip should it be inadvertently retracted through the introducer, as discussed above). The outer diameter of the second straight section 2050c and tapered distal section 2050b′ is preferably less than approximately 0.120″ to allow passage of these sections of the introducer tool 2000 through conventional standard Rotating Hemostasis Valve (RHV) typically used with conventional guide catheters. Whereas, the inner diameter of the first straight section 2050a is preferably sufficiently large to accommodate strain relief of the aspiration catheter (e.g., funnel catheter) therein thereby permitting full insertion of the axial/longitudinal length of the aspiration catheter without having to remove the introducer tool. Also, the inner diameter of the first straight section 2050a of the introducer tool is sufficient to allow for easy loading therein of the flared distal section of the funnel catheter while in a radially uncompressed or uncollapsed flared state; whereas the outer diameter of the first straight section 2050a is sufficiently large to prevent over insertion through the hemostasis valve.

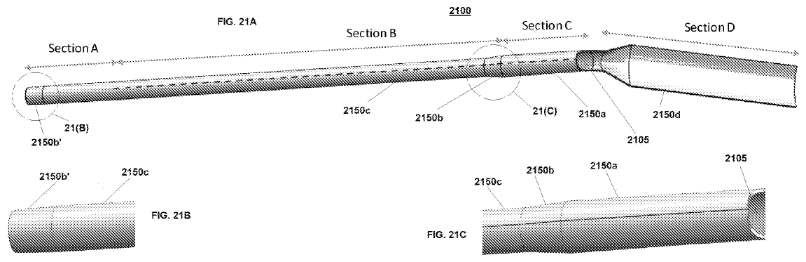

The introducer tool 2100 may be further modified, as shown in FIGS. 21A-E, to be semi (i.e., partially; not completely, entirely or fully to the distal end) split in an axial/longitudinal direction starting from the distal end of the handles 2150d (i.e., the proximal tip/end of the lumen) and terminating proximally of the distal tip/end of the second straight section 2150c. In other words, the semi or partial split (e.g., along a continuous slit or series of perforations) extends in an axial/longitudinal direction the length of section “C” (representing the entire axial/longitudinal length of the first straight section 2150a) and the length of section “B” (representing the entire axial/longitudinal length of the transition section 2150b and only partially the axial/longitudinal length (but not completely to the distal tip/end) of the second straight section 2150c). Introducer tool 2100 includes an unsplit section “A” (i.e., free of any split or separation) including the entire tapered distal section 2150b′ and a distal portion (including the distal tip/end) of the second straight section 2105c. This unsplit section “A” of the introducer tool 2100 maintains sufficient strength to prevent collapse of the inner diameter when its distal end is pushed into the tapered guide sheath luer/hub. Preferably, the axial/longitudinal length of unsplit section “A” of the introducer tool 2100 is approximately 20 mm. In contrast to the example in FIGS. 6A & 6B in which the split extends the entire axial/longitudinal length (i.e., from the proximal end to the distal end) of the introducer tool so that once separated the introducer tool is removable from about the aspirator catheter, in FIG. 21 the introducer tool remains positioned in place about the aspirator catheter wherein the semi-split merely allows for slight radial expansion (i.e., radial accommodation) of the flared distal section of the aspirator catheter while traversing through that portion of the lumen of reduced inner diameter.

With the peelable apart handles/tabs 2150d radially spread apart/bent the aspiration catheter with its flared distal section in a radially uncompressed/uncollapsed state is loaded into the lumen 2105 of the introducer tool 2100 via the first straight section 2150a. The large inner diameter of the first straight section 2150a of the introducer tool 2100 facilitates easy insertion therein of the aspiration catheter with its flared distal section while in a radially uncompressed/uncollapsed state while also providing stability as the flared distal section of the aspiration catheter is radially compressed/collapsed when pushed through the reduced inner diameter of the transition section 2150b and into the second straight section 2150c. As the flared distal section of the aspirator catheter passes through the reduced inner diameter of the lumen 2105 in the transition section 2150b and second straight section 2150c, the split (whether continuous or perforated) allows for slight radial expansion of the introducer tool. This expansion in radial diameter minimizes friction between the components and optimizes easy loading of the aspiration catheter by the interventionalist. Upon reaching the distal end of the transition section 2150b, the flared distal section of the aspirator catheter is sufficiently radially compressed/collapsed to allow its passage through the reduced inner diameter of the second straight section 2150c. Accordingly, the inner diameter (e.g., 0.088″) is maintained through which the aspiration catheter is advanced while its flared distal section is radially compressed/collapsed to be receivable in the lumen of the guide sheath.

Inner diameter of section “C” of the introducer tool is larger than the outer diameter of the radially uncompressed/uncollapsed flared distal section of the aspiration catheter to facilitate easy insertion therein. The split along section “C” allows slight radial expansion of the inner diameter as the radially uncompressed/uncollapsed flared distal section of the funnel catheter passes therethrough facilitating easy insertion. Furthermore, the split along section “C” also provides stability while the distal end of the aspiration catheter is partially radially compressed/collapsed when pushed through the reduced inner diameter of section “B”. While the split in axial/longitudinal length along section “B” of the introducer tool allows for slight or reduced (i.e., less than that in section “C”) radial expansion of the inner diameter along the second straight section 2150c minimizing friction and allowing easy loading by the interventionalist of the flared distal section of the aspiration catheter 2100. Lastly, as the flared distal section of the aspiration catheter 2100 transitions from the slightly expanded inner diameter resulting from the split in sections “B” & “C” into the unsplit section “A” (whose inner diameter is reduced and not radially expandable) the flared distal section of the aspiration catheter is sufficiently radially compressed/collapsed proximal to reaching the distal tapered section 2105b′ sufficient to be received in the inner diameter of the guide sheath lumen.

Preferably, the outer diameter of sections “A” & “B” is <approximately 0.120″ to allow passage through standard RHV valves conventionally used with guide catheters. Whereas, the outer diameter of section “C” of the introducer tool is preferably larger than the outer diameter of the strain relief associated with the aspiration catheter allowing it to pass through, thereby making use of the full effective length of the aspiration catheter without having to remove the introducer tool.

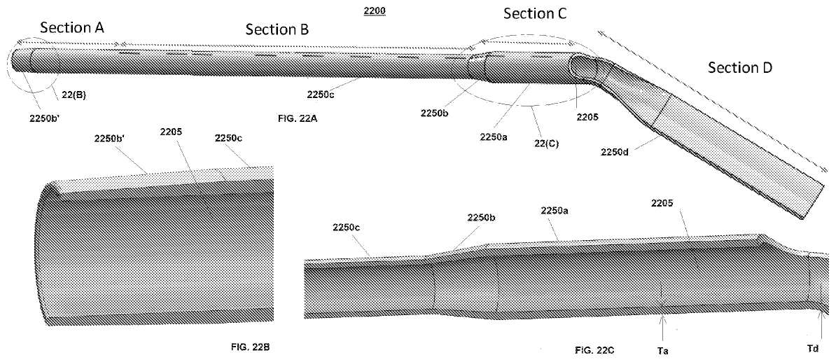

FIGS. 22A-22C depict several views of a still further possible modification of the semi-split introducer tool of FIG. 21 with enhanced rigidity providing greater stability when manipulated or held by the interventionalist. Referring to FIG. 22A, enhanced rigidity of the proximal section is provided by increasing in radial thickness the wall “Td” of the introducer tool along the “D” section (i.e., handles 2250d) and increasing in radial thickness the wall “Ta” along section “C” (i.e., along the first straight section 2250a. Different techniques are available for increasing the thickness of the wall of the introducer tool 2200, such as, by reflowing an extra jacket over the outer diameter or variable thickness extrusion processing. As previously noted with respect to FIGS. 20A & 21A, the outer diameter of the sections “A” and “B” are preferably <approximately 0.120″ to allow passage of the introducer tool through a conventional RHV typically supplied with guide catheters. The tapered distal tip 2250b′ preferably has a tapered outer diameter having an axial/longitudinal length of approximately 1 mm-approximately 3 mm. Also, the edge of the distal end/tip along both the inner and outer diameter is preferably rounded or blunt to minimize scraping off of the coating from the flared distal end of the aspiration catheter when withdrawn/retracted proximally through the introducer tool, as discussed it detail above. Partial axial/longitudinal cross-sectional views of the introducer tool 2200 of FIG. 22A in section 22(B) and section 22(C) are depicted in FIGS. 22B & 22C, respectively. The axial/longitudinal cross-sectional view of FIG. 22B depicts the uniform inner diameter and tapered outer diameter along the tapered distal section 2250b′. Whereas, FIG. 22C illustrates the increased wall thickness “Td” along the handle 2250d of section “D” and the increased wall thickness “Ta” along the first straight section 2250a of section “C”.

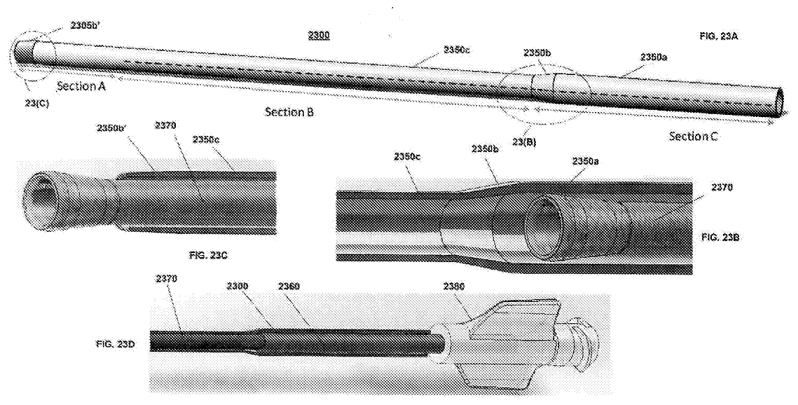

Still another alternative example of the introducer tool in accordance with the present disclosure is shown in FIGS. 23A-23D. This example differs from that of FIGS. 20, 21 & 22 in that it eliminates the need for a proximal section “D” to be divided, radially separated/bent, and flattened to form the handles. Rather a proximal end of the introducer tool 2300 terminates with section “C” providing greater axial/longitudinal length (as a result of eliminating the need for handles) and preferably enhanced rigidity attributed to an increased wall thickness. As a result of the longer length and increased rigidity, the interventionalist is able to manipulate the introducer tool without the need for handles, as in preceding examples shown in FIGS. 20, 21 & 22. Starting from the proximal end/tip, the example introducer tool 2300 in FIG. 23A includes a first straight section 2350a (i.e., cylindrical, uniform in both inner and outer diameter), transition section 2350b having a tapered outer diameter and tapered inner diameter, a second straight section 2350c (i.e., cylindrical, uniform in both inner and outer diameter), followed by a tapered distal section 2350b′ having a uniform inner diameter and a tapered outer diameter distal portion including the distal tip/end). As in FIGS. 20, 21 & 22, here to in the example depicted in FIG. 23A the introducer tool 2300 is semi-split in an axial/longitudinal direction via a series of perforations starting at the proximal end/tip of the lumen 2305 and extending completely through sections “C” & “B” without extending into distal section “A”.

FIG. 23B is an enlarged axial/longitudinal cut away view of a portion of the introducer tool including the transition section 2350b of FIG. 23A illustrating the clearance space between the outer surface of the radially uncompressed/uncollapsed flared distal section of the aspirator catheter 2370 and the inner wall of the first straight section 2350a of the introducer tool 2300 prior to entering transition section 2350b. While traversing through the transition section 2350b and second straight section 2350c, the reduced inner diameter radially compresses/collapses the flared distal section of the aspirator catheter while simultaneously therewith the split in the introducer tool allows for slight (less than that while traversing through the second straight section 2350c) radially expansion, similarly to that described previously with regards to FIGS. 21A & 22A. The flared distal section while in a radially compressed/collapsed state traverses the second straight section 2350c and tapered distal section 2350b′, wherein once again the split allowing for even further radial expansion. Upon exiting out from the distal end/tip of the introducer tool 2300 the radially compressed/collapsed flared distal section of the aspirator catheter 2370 automatically reverts to a radially expanded/uncollapsed/uncompressed state, as illustrated in FIG. 23C depicting an enlarged view of an axial/longitudinal cut away view of section “A” of FIG. 23A.

Since the divided flattened proximal section forming the handles is eliminated in the example of FIG. 23A, the first straight section 2350a of the introducer tool 2300 preferably has an inner diameter sized to allow a clearance fit over the strain relief 2360 when assembled to the proximal end of the aspirator catheter 2370. As a result of the strain relief 2360 being accommodatable in the lumen of the introducer tool 2300, maximum effective insertion in an axial/longitudinal length of the aspiration catheter 2370 is possible, as illustrated in FIG. 23D. Over insertion of the aspirator catheter 2370 into the introducer tool 2300, however, is still prohibited by the proximal hub 2380. This example introducer tool therefore may remain in place for the entire procedure without having to be removed.

These are a few non-limiting examples while still other mechanical structural mechanisms are contemplated and within the scope of the present disclosure that absorb excessive compression force (i.e., over insertion) in an axial/longitudinal direction thereby preventing transfer to the distal end/tip in order to thwart compression of the lumen. Regardless of the mechanical structural mechanism the radially expandable compression force absorption component comprising a portion of the straight (i.e., cylindrical, uniform in both inner and outer diameters) section is disposed between the handle and the outer diameter transition section of the introducer tool. So as not to restrict or limit radial expansion, when the introducer tool is fully inserted into the assembly the radially expandable compression force absorption component is external (i.e., proximal) of the hemostatic valve.

In any of the examples for the introducer tool described herein, the inner profile of the lumen of the introducer tool preferably has a non-circular radial cross-sectional geometry. The non-circular radial cross-sectional geometry of the lumen of the introducer may take on a variety of configurations or designs. In one instance, the non-circular radial cross-sectional geometry of the inner profile of the lumen of the introducer tool has one or more ribs projecting/raised radially inward from the inner wall of the lumen and extending in an axial/longitudinal direction. Extending in an axial/longitudinal direction the internal ribs projecting radially inward from an inner wall of the lumen may be: (i) a continuous (i.e., uninterrupted) pattern/design extending from the proximal end/tip to the opposite distal end/tip or along only a section/portion thereof the introducer tool; or (iii) a non-continuous (i.e., interrupted, broken on/off) pattern/design along more than one axial/longitudinal section/portion thereof the introducer tool with devoid sections/portions therebetween. Any pattern/design of the internal rib is contemplated such as a linear (i.e., straight line) and/or a helical pattern. In addition to the number of ribs, the arrangement thereof (i.e., spacing between adjacent ribs) and dimensions (i.e., axial/longitudinal length, width perpendicular to the axial/longitudinal length, depth (from the inner wall of the lumen in a direction radially inward)) associated therewith may be selected, as desired. Ribs may be arranged 360° along the inner wall/profile/surface of the lumen of the introducer tool or only along a radial portion/section/arc thereof, for example, over a 45°, 90° or 180° thereof.

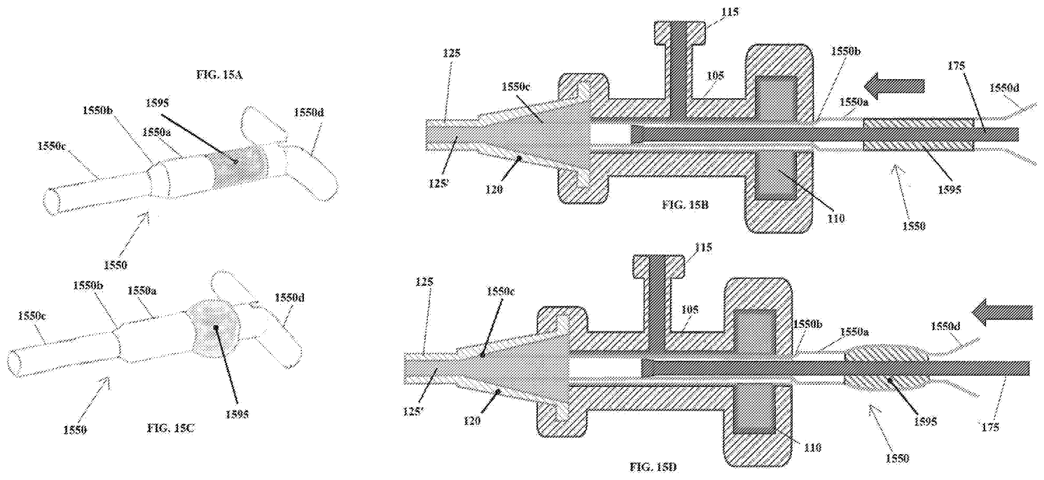

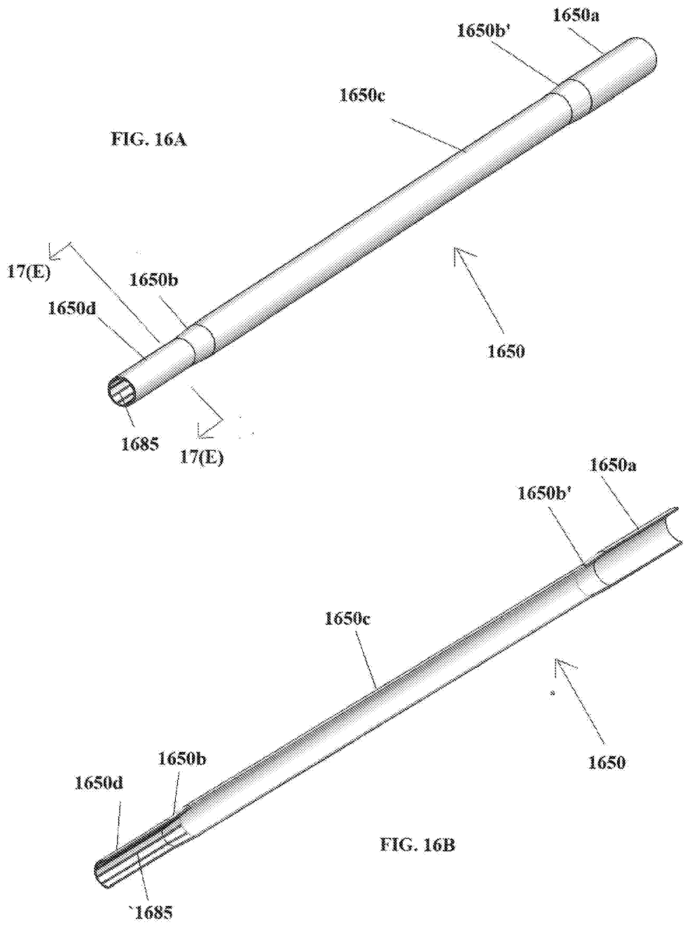

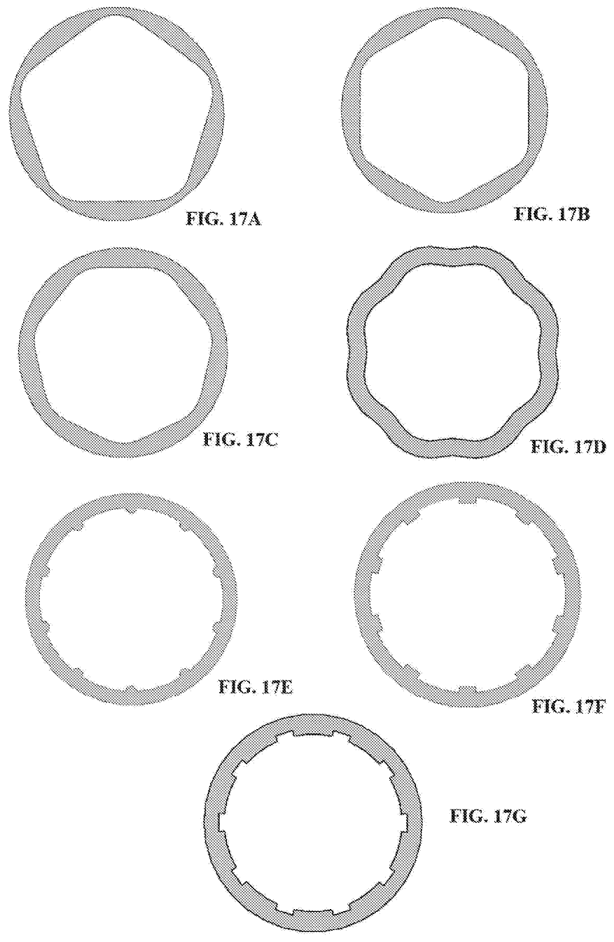

FIG. 16A is a perspective view of the distal end of the introducer tool 1650 similar to introducer tool 850 in FIG. 8A with an enlarged (or flared) proximal section 1650a, a proximal transition section 1650b′ (tapered or stepped controlling insertion depth into the assembly), a straight (cylindrical) section 1650c, a distal transition section 1605b (tapered or stepped), and another straight (cylindrical) extension section 1650d. Introducer tool 1650 in FIG. 16A also includes a plurality of ribs 1685 projecting radially inward from the inner wall/surface of the lumen and extending in an axial/longitudinal direction. A radial cross-sectional view through the raised ribs 1685 along line 17(E)-17(E) is represented in FIG. 17E while FIG. 16B depicts a cutaway view of the distal end perspective view of the introducer tool 1650 in FIG. 16A with a distal section of the outer wall removed clearly illustrating the arrangement of internal ribs 1685 therein.

The raised ribs in FIG. 17E have a semi-circular radial cross-sectional shape arranged equidistantly 360° about the inner wall of the lumen of the introducer tool, whereas in the example in FIG. 17F the raised ribs arranged equidistantly 360° about the inner wall of the lumen of the introducer tool have a square/rectangular radial cross-sectional shape. Other radial cross-sectional geometries of the raised ribs within the lumen of the introducer tool are contemplated and within the intended scope of the disclosure.

Rather than raised ribs in FIG. 17F, the non-circular radial cross-section of the lumen of the introducer tool may have a plurality of recesses/channels extending in a longitudinal/axial direction defined in the inner wall/surface of the lumen of the introducer tool, the radial cross-section of each recess/channel is square/rectangular (FIG. 17G) or any other geometric shape (e.g., semi-circular, triangular, etc.).

The non-circular geometry of the lumen of the introducer tool need not include raised ribs or recesses/channels. By way of non-limiting examples FIGS. 17A-17C depict various multi-sided geometries of the radial cross-section of the lumen of the introducer tool, specifically as a 5-sided, a 6-sided, or a 7-sided geometric shape, respectively. Any other non-circular (e.g., multi-sided geometric) shape of the radial cross-section of the inner profile of the lumen of the introducer is possible. In FIGS. 17A-17C the inner and outer profiles of the introducer tool are not identical. In particular, the outer profile of the introducer tool has a circular radial cross-section, whereas the inner profile of the lumen has a non-circular (e.g., multi-sided geometric shape) radial cross-section. Alternatively, the non-circular inner and outer profiles of the distal section of the introducer tool may conform/identical/match one another, as in the example of FIG. 17D.

Any non-circular radial cross-sectional geometry of the inner profile of the lumen of the introducer tool is possible to provide a radial offset/clearance between an outer profile of the distal section of the funnel catheter while in a flared state and the inner wall of the lumen of the introducer tool serving a dual benefit. In one aspect, the non-circular radial cross-section geometry of the inner profile of the lumen of the introducer tool minimizes surface contact and thus friction between the two components as the aspiration catheter with its distal section in a flared state passes through the lumen of the introducer tool (in comparison to that of an introducer tool having a circular radial cross-sectional geometry of the inner profile lumen). Still another benefit provided by the non-circular radial cross-section geometry of the inner profile of the lumen of the introducer tool is the axial/longitudinal channels/passageways formed by the offset/clearance between the distal section of the aspiration catheter while in a flared state and the inner wall of the introducer tool allowing flow therebetween of fluid (e.g., blood and/or saline) and/or entrained air.

Thus far, the discussion has been directed to the design of the introducer tool itself. The present disclosure also optionally includes a holder suitable for use with any of the introducer tool configuration/examples described herein. The holder divisible axially/longitudinally into two or more components that connectable (e.g., via a snap-fit, magnets, etc.) together as a single unit around/about a proximal portion of the introducer tool that at all times remains outside/exterior of the hemostasis valve during insertion of the introducer tool into the assembly. For example, holder 900 in FIG. 9 comprises two axial/longitudinal half sections 905a, 905b connectable together via complementary, mating or engaging features (e.g., snap-fit). The holder 900 for the introducer tool may comprise more than two axial/longitudinal sections, for example, three, four or more axial/longitudinal sections, as desired. Several benefits are provided by the multi-component holder: (i) enhanced gripping surface by the interventionalist preventing slippage; and (ii) protective supporting structure to the flared distal section (of softer or more pliable material relative to that of the catheter shaft) of the funnel catheter when advanced through the introducer tool; and (iii) readily disassembled for easy removal allowing further advancement/insertion in a distal direction of the funnel catheter length/depth into the assembly; and (iv) since the holder extends in a proximal direction the axial/longitudinal length relative to the proximal end of the introducer tool the holder thereby increases the “lead in” through which the funnel catheter is advanceable.

Referring FIG. 9, the outer profile/surface of the holder 900 depicted has a generally cylindrical shape, but may be modified, as desired, for example to ergonomically have recesses into which the fingers may rest to enhance the grip by the interventionalist. Pieces of the multi-component holder are designed so that when connected together as a unit form a single internal passageway in an axial/longitudinal direction. Starting at the proximal end, the single passageway has a wide tapered proximal entrance to allow the flared distal end in an open biased state of the funnel catheter to be easily introduced unhindered therein. Following the wide tapered proximal entrance, the single passageway has a straight (uniform inner diameter) section whose inner diameter is smaller than the maximum diameter at the proximal end of the passageway, but nevertheless larger than that of the outer diameter of the distal section in a flared (i.e., open biased) state of the funnel catheter allowing it to be advanced unhindered therethrough. Thereafter, the single passageway transitions to a distal portion having a wider inner diameter that thereafter tapers smaller in a distal direction inner diameter matching that of the outer profile of the cone shaped proximal section of the introducer tool accommodatable therein while preventing unintended movement of the introducer tool into the straight section of the single passageway when the interventionalist pushes on the holder in a proximal direction.

Any of the configurations for the introducer tool illustrated and described herein may be modified to include internal ribs arranged on the inner wall of the lumen and/or flushing ports defined therein. It is also noted that any introducer tool may be modified to be split (in accordance with the description of FIGS. 5A-5D) or peel away (in accordance with the description of FIGS. 6A-6D), and such split or peel away may be with, or without, flushing ports. Lastly, the use of the holder as depicted in FIG. 9 and described herein may be employed with any configuration of the introducer tool disclosed and illustrated herein.

### Example 1

A vascular entryway system comprising: an assembly comprising: a guide sheath catheter (125) having a proximal end and a lumen (125′); a tapered guide sheath lure (120) having a proximal end, an opposite distal end, and a tapered inner profile; the proximal end of the guide sheath catheter (125) is received in the distal end of the tapered guide sheath luer (120); a hemostasis valve (105) having a proximal end and an opposite distal end; the proximal end of the tapered guide sheath luer (120) is connected to the distal end of the hemostasis valve (105); an introducer tool (150, 250, 350, 450, 550, 650, 750, 850, 1050, 1150, 1250, 1350, 1450, 1550, 1650, 2000, 2100, 2200, 2300) including a shaft having a proximal end, a distal section including a distal end, a lumen extending axially from the proximal end to the distal end of the shaft, the distal end of the shaft of the introducer tool (150, 250, 350, 450, 550, 650, 750, 850, 1050, 1150, 1250, 1350, 1450, 1550, 1650, 2000, 2100, 2200, 2300) is inserted in the hemostasis valve (105) and the tapered guide sheath luer (120) of the assembly; and an aspiration catheter (175) including a shaft with a radially self-expanding distal section transitionable from a radially non-compressed state of maximum outer diameter to a radially compressed state having a reduced outer diameter; the aspiration catheter (175) is advanceable through the lumen of the introducer tool (150, 250, 350, 450, 550, 650, 750, 850, 1050, 1150, 1250, 1350, 1450, 1550, 1650, 2000, 2100, 2200, 2300) and into the lumen (125′) of the guide sheath catheter (125); wherein at least a portion of the lumen of the shaft of the introducer tool (150, 250, 350, 450, 550, 650, 750, 850, 1050, 1150, 1250, 1350, 1450, 1550, 1650, 2000, 2100, 2200, 2300) has an inner diameter greater than that of the radially self-expanding distal section of the aspiration catheter (175) while in the radially non-compressed state of maximum outer diameter; and wherein the lumen of the shaft of the introducer tool (150, 250, 350, 450, 550, 650, 750, 850, 1050, 1150, 1250, 1350, 1450, 1550, 1650, 2000, 2100, 2200, 2300) and/or the tapered inner profile of the tapered guide sheath luer (120) includes a compressing section with an inner diameter smaller than that of the radially self-expanding distal section of the aspiration catheter (175) while in the radially non-compressed state of maximum outer diameter.

### Example 2

The system of Example 1, wherein the distal section of the shaft of the introducer tool (250, 350, 450, 550, 650, 650′, 750, 2000, 2100, 2200, 2300) has a tapered outer diameter (250b, 350b, 450b, 550b, 650b, 650′b, 750b, 2050b′, 2150b′, 2250b′, 2350b′) matching the tapered inner profile of the tapered guide sheath luer (120).

### Example 3

The system of any of Examples 1 through 2, wherein the aspiration catheter (175) has a flared proximal section (175′a) conforming in shape and size with a flared proximal section (750a) of the introducer tool (750) so that when fully inserted is nestable therein.

### Example 4

A method of using an vascular entryway system including: an assembly comprising: a guide sheath catheter (125) having a proximal end and a lumen (125′); a tapered guide sheath lure (120) having a proximal end, an opposite distal end, and a tapered inner profile; the proximal end of the guide sheath catheter (125) is received in the distal end of the tapered guide sheath luer (120); and a hemostasis valve (105) having a proximal end and an opposite distal end, the proximal end of the tapered guide sheath luer (120) is connected to the distal end of the hemostasis valve (105); the system further including an introducer tool (150, 250, 350, 450, 550, 650, 750, 850, 1050, 1150, 1250, 1350, 1450, 1550, 1650, 2000, 2100, 2200, 2300) including a shaft having a proximal end, a distal section including a distal end, and a lumen; the distal end of the shaft of the introducer tool (150, 250, 350, 450, 550, 650, 750, 850, 1050, 1150, 1250, 1350, 1450, 1550, 1650, 2000, 2100, 2200, 2300) being advanceable through the hemostasis valve (105) and into the tapered guide sheath luer (120) of the assembly; and the system also including an aspiration catheter (175) including a shaft with a radially self-expanding distal section transitionable from a radially non-compressed state of maximum outer diameter to a radially compressed state having a reduced outer diameter; the aspiration catheter (175) is advanceable through the lumen of the introducer tool (150, 250, 350, 450, 550, 650, 750, 850, 1050, 1150, 1250, 1350, 1450, 1550, 1650, 2000, 2100, 2200, 2300) and into the lumen (125′) of the guide sheath catheter (125); wherein at least a portion of the lumen of the shaft of the introducer tool (150, 250, 350, 450, 550, 650, 750, 850, 1050, 1150, 1250, 1350, 1450, 1550, 1650, 2000, 2100, 2200, 2300) has an inner diameter greater than that of the radially self-expanding distal section of the aspiration catheter (175) while in the radially non-compressed state of maximum outer diameter; and wherein the lumen of the shaft of the introducer tool (150, 250, 350, 450, 550, 650, 750, 850, 1050, 1150, 1250, 1350, 1450, 1550, 1650, 2000, 2100, 2200, 2300) and/or the tapered inner profile of the tapered guide sheath luer (120) includes a compressing section with an inner diameter smaller than that of the radially self-expanding distal section of the aspiration catheter (175) while in the radially non-compressed state of maximum outer diameter; the method comprising the steps of: while the radially self-expanding distal section of the aspiration catheter (175) is in the radially non-compressed state of maximum outer diameter, pre-assembling the aspiration catheter (175) into a proximal section of the introducer tool (150, 250, 350, 450, 550, 650, 750, 850, 1050, 1150, 1250, 1350, 1450, 1550, 1650, 2000, 2100, 2200, 2300); together as a single unit, introducing the introducer tool (150, 250, 350, 450, 550, 650, 750, 850, 1050, 1150, 1250, 1350, 1450, 1550, 1650, 2000, 2100, 2200, 2300) with the aspiration catheter (175) pre-assembled therein into the hemostasis valve (105) and the tapered guide sheath luer (120) of the assembly; pushing the aspiration catheter (175) through the introducer tool (150, 250, 350, 450, 550, 650, 750, 850, 1050, 1150, 1250, 1350, 1450, 1550, 1650, 2000, 2100, 2200, 2300); and while traversing the compressing section of the lumen of the shaft of the introducer tool (150, 250, 350, 450, 550, 650, 750, 850, 1050, 1150, 1250, 1350, 1450, 1550, 1650, 2000, 2100, 2200, 2300) and/or the tapered inner profile of the tapered guide sheath luer (120), radially compressing the radially self-expanding distal section of the aspiration catheter (175) sufficient to be receivable in the lumen (125′) of the guide sheath catheter (125); and while the radially self-expanding distal section is radially compressed, sliding the aspiration catheter (175) into the lumen (125′) of the guide sheath catheter (125).

### Example 5

The method of Example 4, wherein the pushing step further comprises the step of automatically hydrating a hydrophilic coating of the aspiration catheter (175) by back pressure of blood passing in a proximal direction within a clearance space defined between the inner diameter of the lumen of the shaft of the introducer tool (150, 250, 350, 450, 550, 650, 750, 850, 1050, 1150, 1250, 1350, 1450, 1550, 1650, 2000, 2100, 2200, 2300) and an outer profile of the radially self-expanding distal section of the aspiration catheter (175) while in the radially non-compressed state of maximum outer diameter.

### Example 6

The method of any of Examples 4 through 5, wherein the shaft of the introducer tool (450, 550, 650′) has a plurality of flushing ports (455, 555, 655) defined therein; and during the step of pushing the aspiration catheter (175) through the introducer tool (450, 550, 650′) while assembled in the hemostasis valve (105), producing a back pressure of blood in a proximal direction and entrained air passing through the plurality of flushing ports (455, 555, 655) and exiting in the proximal direction from the introducer tool (450, 550, 650′).

### Example 7

The method of Example 6, wherein the hemostasis valve (450, 550, 650′) has a side port (115); and the method further comprising the step of positive flushing of the lumen of the shaft of the introducer tool (450, 550, 650′) with a fluid injected via the side port (115) of the hemostasis valve (105) passing through the plurality of flushing ports (455, 555, 655).

### Example 8

The method of any of Examples 4 through 7, further comprising the step of removing the introducer tool (550, 550′, 550″) from around the aspiration catheter (175) via a longitudinal slit (560, 560′, 560″) defined therein extending from the proximal end to the distal end of the introducer tool allowing further insertion in a distal direction of the aspiration catheter (175) into the assembly.

### Example 9

The method of any of Examples 4 through 7, wherein the shaft of the introducer tool (2100, 2200, 2300) includes a semi-split section extending in a longitudinal direction from a proximal end of the lumen and terminating proximally of the distal end of the introducer tool (2100, 2200, 2300) defining distally thereof an unsplit distal section; and wherein, when radially compressed while traversing the compressing section of the lumen of the shaft, the introducer tool (2100, 2200, 2300) radially expanding along the semi-split section to accommodate the radially self-expanding distal section of the aspiration catheter (175).

### Example 10

The method of any of Examples 4 through 9, wherein the introducing step comprises limiting a depth in which the introducer tool is insertable into the tapered guide sheath lure (120) of the assembly to prevent axial compression and narrowing of the lumen of the distal end of the shaft of the introducer tool when engaging with the tapered inner profile of the tapered guide sheath luer (120).

### Example 11

The method of any of Examples 4 through 10, wherein the shaft of the introducer tool includes: a distal section including the distal end of the shaft of the introducer tool; a proximal section including the proximal end of the shaft of the introducer tool; a transition section disposed between the distal and proximal sections of the shaft of the introducer tool; wherein each of the distal, proximal and transition sections of the shaft of the introducer tool are receivable within the passageway of the hemostasis valve (105); and wherein the pushing step comprises traversing the proximal section of the shaft of the introducer tool while maintaining the radially self-expanding distal section of the aspiration catheter (175) in the radially non-compressed state of maximum outer diameter and radially compressing the radially self-expanding distal section of the aspiration catheter (175) while passing through the distal section of the shaft of the introducer tool.

### Example 12

The method of any of Examples 4 through 11, wherein the shaft of the introducer tool from the proximal end to the distal end has a tapered inner diameter and a tapered outer diameter; and the pushing step comprises entering the proximal end of the shaft of the introducer tool while maintaining the radially self-expanding distal section of the aspiration catheter (175) in the non-compressed state and radially compressing the radially self-expanding distal section of the aspiration catheter (175) while emerging from the distal end of the shaft of the introducer tool.

### Example 13

The method of any of Examples 4 through 12, wherein the pushing step comprises the steps of: securing a holder (900) including a plurality of components connectable together defining therein a channel accommodating therein the proximal section of the introducer tool and the aspiration catheter (175) pre-assembled therein; and gripping the holder (900) while pushing the aspiration catheter (175) through the introducer tool (350) and into the assembly; and wherein the pushing step further comprises removing the holder (900) from the aspiration catheter (175) allowing further insertion into the assembly.

### Example 14

The method of any of Examples 4 through 13, wherein the introducing step further comprises preventing compression and narrowing of the lumen at the distal end of the introducer tool when over inserting the introducer tool (1150, 1250, 1350, 1450, 1550) into the assembly by deploying a radially expandable compression force absorption component (1195, 1295, 1395, 1495, 1595) associated with the shaft of the introducer tool; wherein the radially expandable compression force absorption component is (1195, 12,95, 1395, 1495, 1595): (i) a plurality of radially expanding pleats; (ii) one or more radially expandable sections in the shaft of the introducer tool having a plurality of slits defined therein; or (iii) a section of material of reduced stiffness relative to that of the rest of the introducer tool.

### Example 15

The method of any of Examples 4 through 14, wherein the shaft of the introducer tool includes an axially non-contractable section (1850, 1950b) disposed distally of an axially contractable section (1850′, 1950c); wherein the axially contractable section (1850′, 1950c) is transitionable from a state of maximum axial length to a state of reduced axial length; and wherein the radially compressing step comprises reducing the radially self-expanding distal section of the aspiration catheter (175) so as to be receivable in the lumen (125′) of the guide sheath catheter (125) as the aspiration catheter (175) passes through the axially contractable section (1850′, 1950c) of the shaft of the introducer while in the state of maximum axial length.

### Example 16

A vascular introducer tool (850, 850′, 2000, 2100, 2200, 2300) comprising: a shaft having an outer wall extending from a proximal end to an opposite distal end with a lumen extending in a longitudinal direction defined therethrough; and disposed between the proximal and distal ends the shaft including an intermediate transition section (850b, 850′b, 2050b, 2150b, 2250b, 2350b) having a tapered inner diameter and a tapered outer diameter.

### Example 17

The vascular introducer tool of Example 16, wherein the outer wall of the shaft has a plurality of flushing ports defined therein in fluid communication with the lumen.

### Example 18

The vascular introducer tool of any of Examples 16 through 17, wherein the outer wall of the shaft is splitable in the longitudinal direction from the proximal end to the opposite distal end along one of: (i) a slit as defined by two longitudinal edges; or (ii) a weakened section.

### Example 19

The vascular introducer tool of any of Examples 16 through 18, wherein the outer wall of the shaft includes a semi-split section extending in a longitudinal direction from a proximal end of the lumen and terminating proximally of the distal end of the introducer tool defining distally thereof an unsplit distal section.

### Example 20

The vascular introducer tool of any of Examples 16 through 19, wherein a proximal section of the outer wall of the shaft is splitable in the longitudinal direction into multiple proximal divided sections separated away from one another relative to a longitudinal axis through the vascular introducer tool forming respective tabs (2050d, 2150d, 2250d).

### Example 21

The vascular introducer tool of any of Examples 16 through 20, wherein the shaft has a lumen extending therethrough from the proximal to the distal end, wherein the lumen of the shaft of the introducer tool has a non-circular shape radial cross-section; wherein the non-circular shape radial cross-section includes: (i) a plurality of ribs (1685) projecting radially inward from an inner wall of the lumen of the shaft of the introducer tool and extending in a longitudinal direction; or (ii) a plurality of recesses defined in the inner wall of the lumen of the shaft of the introducer tool and extending in the longitudinal direction.

Thus, while there have been shown, described, and pointed out fundamental novel features of the introducer tool for an aspiration catheter, it will be understood that various omissions, substitutions, and changes in the form and details of the systems/devices illustrated, and in their operation, may be made by those skilled in the art without departing from the spirit and scope of the disclosure. For example, it is expressly intended that all combinations of those elements and/or steps that perform substantially the same function, in substantially the same way, to achieve the same results be within the scope of the disclosure. Substitutions of elements from one described example to another are also fully intended and contemplated. It is also to be understood that the drawings are not necessarily drawn to scale, but that they are merely conceptual in nature. It is the intention, therefore, to be limited only as indicated by the scope of the claims appended hereto.

Every issued patent, pending patent application, publication, journal article, book or any other reference cited herein is each incorporated by reference in their entirety.

What is claimed is:

1. A vascular entryway system comprising:
an assembly comprising:
a guide sheath catheter having a proximal end and a lumen;
a tapered guide sheath lure having a proximal end, an opposite distal end, and a tapered inner profile; the proximal end of the guide sheath catheter is received in the distal end of the tapered guide sheath luer;
a hemostasis valve having a proximal end and an opposite distal end; the proximal end of the tapered guide sheath luer is connected to the distal end of the hemostasis valve;
an introducer tool including a shaft having a proximal end, a distal section including a distal end, a lumen extending axially from the proximal end to the distal end of the shaft, the distal end of the shaft of the introducer tool is inserted in the hemostasis valve and the tapered guide sheath luer of the assembly; and
an aspiration catheter including a shaft with a radially self-expanding distal section transitionable from a radially non-compressed state of maximum outer diameter to a radially compressed state having a reduced outer diameter; the aspiration catheter is advanceable through the lumen of the introducer tool and into the lumen of the guide sheath catheter;
wherein at least a portion of the lumen of the shaft of the introducer tool has an inner diameter greater than that of the radially self-expanding distal section of the aspiration catheter while in the radially non-compressed state of maximum outer diameter; and
wherein the lumen of the shaft of the introducer tool and/or the tapered inner profile of the tapered guide sheath luer includes a compressing section with an inner diameter smaller than that of the radially self-expanding distal section of the aspiration catheter while in the radially non-compressed state of maximum outer diameter.

2. The system in accordance with claim 1, wherein the distal section of the shaft of the introducer tool has a tapered outer diameter matching the tapered inner profile of the tapered guide sheath luer.

3. The system in accordance with claim 1, wherein the aspiration catheter has a flared proximal section conforming in shape and size with a flared proximal section of the introducer tool so that when fully inserted is nestable therein.

4. A method of using an vascular entryway system including: an assembly comprising: a guide sheath catheter having a proximal end and a lumen; a tapered guide sheath lure having a proximal end, an opposite distal end, and a tapered inner profile; the proximal end of the guide sheath catheter is received in the distal end of the tapered guide sheath luer; and a hemostasis valve having a proximal end and an opposite distal end, the proximal end of the tapered guide sheath luer is connected to the distal end of the hemostasis valve; the system further including an introducer tool including a shaft having a proximal end, a distal section including a distal end, and a lumen; the distal end of the shaft of the introducer tool being advanceable through the hemostasis valve and into the tapered guide sheath luer of the assembly; and the system also including an aspiration catheter including a shaft with a radially self-expanding distal section transitionable from a radially non-compressed state of maximum outer diameter to a radially compressed state having a reduced outer diameter; the aspiration catheter is advanceable through the lumen of the introducer tool and into the lumen of the guide sheath catheter; wherein at least a portion of the lumen of the shaft of the introducer tool has an inner diameter greater than that of the radially self-expanding distal section of the aspiration catheter while in the radially non-compressed state of maximum outer diameter; and wherein the lumen of the shaft of the introducer tool and/or the tapered inner profile of the tapered guide sheath luer includes a compressing section with an inner diameter smaller than that of the radially self-expanding distal section of the aspiration catheter while in the radially non-compressed state of maximum outer diameter; the method comprising the steps of:
while the radially self-expanding distal section of the aspiration catheter is in the radially non-compressed state of maximum outer diameter, pre-assembling the aspiration catheter into a proximal section of the introducer tool;
together as a single unit, introducing the introducer tool with the aspiration catheter pre-assembled therein into the hemostasis valve and the tapered guide sheath luer of the assembly;
pushing the aspiration catheter through the introducer tool; and
while traversing the compressing section of the lumen of the shaft of the introducer tool and/or the tapered inner profile of the tapered guide sheath luer, radially compressing the radially self-expanding distal section of the aspiration catheter sufficient to be receivable in the lumen of the guide sheath catheter; and
while the radially self-expanding distal section is radially compressed, sliding the aspiration catheter into the lumen of the guide sheath catheter.

5. The method in accordance with claim 4, wherein the pushing step further comprises the step of automatically hydrating a hydrophilic coating of the aspiration catheter by back pressure of blood passing in a proximal direction within a clearance space defined between the inner diameter of the lumen of the shaft of the introducer tool and an outer profile of the radially self-expanding distal section of the aspiration catheter while in the radially non-compressed state of maximum outer diameter.

6. The method in accordance with claim 4, wherein the shaft of the introducer tool has a plurality of flushing ports defined therein; and during the step of pushing the aspiration catheter through the introducer tool while assembled in the hemostasis valve, producing a back pressure of blood in a proximal direction and entrained air passing through the plurality of flushing ports and exiting in the proximal direction from the introducer tool.

7. The method in accordance with claim 6, wherein the hemostasis valve has a side port; and the method further comprising the step of positive flushing of the lumen of the shaft of the introducer tool with a fluid injected via the side port of the hemostasis valve passing through the plurality of flushing ports.

8. The method in accordance with claim 4, further comprising the step of removing the introducer tool from around the aspiration catheter via a longitudinal slit defined therein extending from the proximal end to the distal end of the introducer tool allowing further insertion in a distal direction of the aspiration catheter into the assembly.

9. The method in accordance with claim 4, wherein the shaft of the introducer tool includes a semi-split section extending in a longitudinal direction from a proximal end of the lumen and terminating proximally of the distal end of the introducer tool defining distally thereof an unsplit distal section; and wherein, when radially compressed while traversing the compressing section of the lumen of the shaft, the introducer tool radially expanding along the semi-split section to accommodate the radially self-expanding distal section of the aspiration catheter.

10. The method in accordance with claim 4, wherein the introducing step comprises limiting a depth in which the introducer tool is insertable into the tapered guide sheath lure of the assembly to prevent axial compression and narrowing of the lumen of the distal end of the shaft of the introducer tool when engaging with the tapered inner profile of the tapered guide sheath luer.

11. The method in accordance with claim 4, wherein the shaft of the introducer tool includes: a distal section including the distal end of the shaft of the introducer tool; a proximal section including the proximal end of the shaft of the introducer tool; a transition section disposed between the distal and proximal sections of the shaft of the introducer tool; wherein each of the distal, proximal and transition sections of the shaft of the introducer tool are receivable within the passageway of the hemostasis valve; and wherein the pushing step comprises traversing the proximal section of the shaft of the introducer tool while maintaining the radially self-expanding distal section of the aspiration catheter in the radially non-compressed state of maximum outer diameter and radially compressing the radially self-expanding distal section of the aspiration catheter while passing through the distal section of the shaft of the introducer tool.

12. The method in accordance with claim 4, wherein the shaft of the introducer tool from the proximal end to the distal end has a tapered inner diameter and a tapered outer diameter; and the pushing step comprises entering the proximal end of the shaft of the introducer tool while maintaining the radially self-expanding distal section of the aspiration catheter in the non-compressed state and radially compressing the radially self-expanding distal section of the aspiration catheter while emerging from the distal end of the shaft of the introducer tool.

13. The method in accordance with claim 4, wherein the pushing step comprises the steps of:
securing a holder including a plurality of components connectable together defining therein a channel accommodating therein the proximal section of the introducer tool and the aspiration catheter pre-assembled therein; and
gripping the holder while pushing the aspiration catheter through the introducer tool and into the assembly; and
wherein the pushing step further comprises removing the holder from the aspiration catheter allowing further insertion into the assembly.

14. The method in accordance with claim 4, wherein the introducing step further comprises preventing compression and narrowing of the lumen at the distal end of the introducer tool when over inserting the introducer tool into the assembly by deploying a radially expandable compression force absorption component associated with the shaft of the introducer tool; wherein the radially expandable compression force absorption component is: (i) a plurality of radially expanding pleats; (ii) one or more radially expandable sections in the shaft of the introducer tool having a plurality of slits defined therein; or (iii) a section of material of reduced stiffness relative to that of the rest of the introducer tool.

15. The method in accordance with claim 4, wherein the shaft of the introducer tool includes an axially non-contractable section disposed distally of an axially contractable section; wherein the axially contractable section is transitionable from a state of maximum axial length to a state of reduced axial length; and wherein the radially compressing step comprises reducing the radially self-expanding distal section of the aspiration catheter so as to be receivable in the lumen of the guide sheath catheter as the aspiration catheter passes through the axially contractable section of the shaft of the introducer while in the state of maximum axial length.

16. A vascular introducer tool comprising:
a shaft having an outer wall extending from a proximal end to an opposite distal end with a lumen extending in a longitudinal direction defined therethrough; and disposed between the proximal and distal ends the shaft including an intermediate transition section having a tapered inner diameter and a tapered outer diameter.

17. The vascular introducer tool in accordance with claim 16, wherein the outer wall of the shaft has a plurality of flushing ports defined therein in fluid communication with the lumen.

18. The vascular introducer tool in accordance with claim 16, wherein the outer wall of the shaft is splitable in the longitudinal direction from the proximal end to the opposite distal end along one of: (i) a slit as defined by two longitudinal edges; or (ii) a weakened section.

19. The vascular introducer tool in accordance with claim 16, wherein the outer wall of the shaft includes a semi-split section extending in a longitudinal direction from a proximal end of the lumen and terminating proximally of the distal end of the introducer tool defining distally thereof an unsplit distal section.

20. The vascular introducer tool in accordance with claim 16, wherein a proximal section of the outer wall of the shaft is splitable in the longitudinal direction into multiple proximal divided sections separated away from one another relative to a longitudinal axis through the vascular introducer tool forming respective tabs.

21. The vascular introducer tool in accordance with claim 16, wherein the shaft has a lumen extending therethrough from the proximal to the distal end, wherein the lumen of the shaft of the introducer tool has a non-circular shape radial cross-section; wherein the non-circular shape radial cross-section includes: (i) a plurality of ribs projecting radially inward from an inner wall of the lumen of the shaft of the introducer tool and extending in a longitudinal direction; or (ii) a plurality of recesses defined in the inner wall of the lumen of the shaft of the introducer tool and extending in the longitudinal direction.
